(19) United States
(12) Patent Application Publication
(10) Pub. No.: US 2007/0093463 A1
(43) Pub. Date: Apr. 26, 2007

(54) Radiation enhancement agent for X-ray radiation therapy and boron neutron-capture therapy

(75) Inventors: Michiko Miura, Hampton Bays, NY (US); Haitao Wu, Rockville, MD (US)

Correspondence Address: BROOKHAVEN SCIENCE ASSOCIATES/;BROOKHAVEN NATIONAL LABORATORY, BLDG. 475D - P.O. BOX 5000, UPTON, NY, 11973, US

(73) Assignee: Brookhaven Science Associates, LLC, Upton, NY (US)

(21) Appl. No.: 11/606,864

(22) Filed: Dec. 1, 2006

Related U.S. Application Data
(63) Continuation-in-part of application No. 10/848,741, filed on May 20, 2004, now Pat. No. 6,995,260.
(63) Continuation-in-part of application No. 10/878,138, filed on Jun. 28, 2004, now Pat. No. 6,989,443.

(30) Foreign Application Priority Data
May 17, 2005 (WO) PCT/US05/17358
Jun. 22, 2005 (WO) PCT/US05/22061

(51) Int. Cl. A61K 31/555; C07D 487/22

(52) U.S. Cl. 514/185; 540/145

(57) ABSTRACT

Low toxicity halogenated carborane-containing tetraphenylporphyrin compounds and methods for their use particularly in boron neutron capture therapy (BNCT), X-ray radiation therapy (XRT) and photodynamic therapy (PDT) for the treatment of tumors of the brain, head and neck, and surrounding tissue. The invention also includes methods of tumor imaging and/or diagnosis such as MRI, SPECT, or PET using these halogenated carborane-containing tetraphenylporphyrin compounds.

This application is a continuation-in-part of International Patent Application No. PCT/US2005/017358, filed on May 17, 2005, which claims priority based on U.S. patent application Ser. No. 10/848,741, filed on May 20, 2004 and issued on Feb. 7, 2006 as U.S. Pat. No. 6,995,260 B2. This application is also a continuation-in-part of International Patent Application No. PCT/US2005/022061, filed on Jun. 22, 2005, which claims priority based on U.S. patent application Ser. No. 10/878,138, filed on Jun. 28, 2004 and issued on Jan. 24, 2006 as U.S. Pat. No. 6,989,443 B2. All of these references are incorporated herein in their entirety.

This invention was made with Government support under contract number DE-AC02-98CH10886, awarded by the U.S. Department of Energy. The Government has certain rights in the invention.

### BACKGROUND OF INVENTION

The present invention relates to low toxicity, halogenated carborane-containing tetraphenylporphyrin compounds and methods for their use particularly in boron neutron capture therapy (BNCT), X-ray radiation therapy (XRT) and photodynamic therapy (PDT) for the treatment of tumors of the brain, head and neck, and surrounding tissue. In particular, the present invention relates to halogenated carborane-containing tetraphenylporphyrin compounds that have greater tumor control with a high therapeutic ratio with regard to normal tissues. The invention also includes methods of tumor imaging and/or diagnosis such as MRI, SPECT, or PET using these halogenated carborane-containing tetraphenylporphyrin compounds.

The efficacy of radiation and chemical methods in the treatment of cancers has been limited by a lack of selective targeting of tumor cells by the therapeutic agent. In an effort to spare normal tissue, current tumor treatment methods have therefore restricted radiation and/or chemical treatment doses to levels that are well below optimal or clinically adequate. Thus, designing compounds that are capable, either alone or as part of a therapeutic method, of selectively targeting and destroying tumor cells, is a field of intense study.

Radiosensitizers are substances that make a cancer cell more susceptible to the effects of radiation therapy, thereby boosting the effect of the radiation dose. When cancers are treated using radiotherapy, the presence of hypoxic cells in the tumor is the greatest problem. Hypoxic tumor cells are resistant to radiation and existing chemotherapy techniques. In contrast to cancerous tumors, normal tissues do not have any hypoxic cells. Accordingly, radiotherapy for treating cancer is more effective when the radiosensitivity of the hypoxic cells in the tumor is enhanced by introducing a radiosensitizer. Attempts have been made to increase the radiosensitivity of hypoxic cells using different compounds, such as nitroimidazoles, as radiosensitizers but the results have been mixed.

Porphyrins in general belong to a class of colored, aromatic tetrapyrrole compounds, some of which are found naturally in plants and animals, e.g., chlorophyll and heme, respectively. Porphyrins are known to have a high affinity to neoplastic tissues of mammals, including man. Because of their affinity for neoplastic tissues, in general, porphyrins with boron-containing substituents can be useful in the treatment of primary and metastatic tumors of the central nervous system by boron neutron capture therapy (BNCT). Porphyrins and other tetrapyrroles with relatively long singlet lifetimes have already been used to treat malignant tumors with photodynamic therapy (PDT), but such use has had limited clinical applicability because of the poor penetration of the visible light required to activate the administered enhancer so as to render it toxic to living tissues, i.e., the targeted tumor.

Porphyrins have the added advantage of being useful in vivo as chelating agents for certain paramagnetic metal ions to achieve higher contrast in magnetic resonance imaging (MRI). They can also be chelated with radioactive metal ions for tumor imaging in single-photon-emission computed tomography (SPECT) or position emission tomography (PET). In principle, porphyrins can also be used for high-specific-activity radioisotope therapy when the carrier molecule can be targeted with sufficient biospecificity to the intended lesion so as to avoid normal tissue radiotoxicity, which is most often encountered, when present at all, in the bladder, bone marrow, liver, and lung—the likely sites of undesired bioaccumulation of unbound carrier or its degradation products.

Boron neutron-capture therapy (BNCT) is a bimodal cancer treatment based on the selective accumulation of a ¹⁰B carrier in tumors, and subsequent irradiation with thermalized neutrons. The production of microscopically localized high linear-energy-transfer (LET) radiation from capture of thermalized neutrons by ¹⁰B in the ¹⁰B(n, α)⁷ Li reaction is responsible for the high efficacy and sparing of normal tissues. More specifically, the stable nuclide ¹⁰B absorbs a thermalized neutron to create two mutually recoiling ionizing high-energy charged particles, ⁷Li and ⁴He, with microscopic ranges of 5 μm and 9 μm, respectively.

When BNCT is used to treat patients with malignant tumors, the patient is given a boron compound highly enriched (≈95 atom %) in boron-10. The boronated compound is chosen based on its ability to concentrate preferentially in the tumor within the radiation volume. In the case of brain tumors, after injection of the boron compound, the patient's head is irradiated in the general area of the brain tumor with an incident beam or field of epithermal (0.5 eV-10 keV) neutrons. These neutrons become progressively thermalized (average energy approximately 0.04 eV) as they penetrate deeper into the head. As the neutrons become thermalized, they can more readily be captured by the boron-10 concentrated in the tumor cells and/or tumor supporting tissues, since the capture cross section is inversely proportional to the neutron velocity. A minuscule proportion of the boron-10 nuclei in and around a tumor undergoes a nuclear reaction immediately after capturing a neutron, which is why such a large concentration of boron-10 is required in and/or around a targeted cell or tissue for BNCT to be clinically effective. The present invention, when implemented clinically alone or in combination with existing or other new therapies, will meet this ‘high-concentration without undue toxicity’ requirement better than previously known compounds. This nuclear reaction produces the high LET alpha (⁴He) and lithium (⁷Li) particles. The tumor in which the boron-10 is concentrated is irradiated by these short range particles which, on average, travel a distance comparable to, or slightly less than, the diameter of a typical tumor cell. Therefore, a very localized, specific reaction takes place whereby the tumor receives a large radiation dose compared with that received by surrounding non-neoplastic tissues, with relatively low boron-10 concentrations.

For BNCT of malignant brain tumors, it is particularly important that there be robust uptake of boron in tumor relative to normal tissues (i.e., blood and normal brain tissues) within the neutron-irradiated target volume. BNCT was used clinically at the Brookhaven National Laboratory Medical Department using p-boronophenylalanine (BPA) as the boron carrier (Chanana et al., Neurosurgery, 44, 1182-1192, 1999). BPA has the outstanding quality of not eliciting any chemical toxicity associated with its usage. However, because the brain and blood boron concentrations are approximately one-third that found in tumor, the tumor dose is restricted. In order to improve upon the currently used boron delivery agent, BPA, it has been postulated that tumor boron concentrations should be greater than 30 μg B/g and tumor:blood and tumor:brain boron ratios should be greater than 5:1 (Fairchild and Bond, Int. J. Radiat. Oncol. Biol. Phys., 11, 831-840, 1985, Miura, et al., Int. J. Cancer, 68, 114-119, 1996).

In XRT of malignant tumors, whereby a radiation enhancement drug is used, the patient is first injected or infused with a radiosensitizing drug. As with BNCT, the drug preferentially localizes in a patient's tumor within the irradiation volume. After a certain period of time, the tumor is then irradiated with a single or multiple fractions of X-rays. The single fraction would involve radiosurgical techniques such as gamma knife.

In PDT of malignant tumors using porphyrins, the patient is injected with a photosensitizing porphyrin drug. The drug localizes preferentially in the tumor within the irradiation volume. The patient's tissues in the zone of macroscopic tumor is then irradiated with a beam of red laser light. The vascular cells of the irradiated tumor and some of the tumor cells are rendered incapable of mitotic activity or may be rendered nonviable outright if the light penetrates the tissue sufficiently. The biochemical mechanism of cell damage in PDT is believed to be mediated largely by singlet oxygen. Singlet oxygen is produced by transfer of energy from the light-excited porphyrin molecule to an oxygen molecule. The resultant singlet oxygen is highly reactive chemically and is believed to react with and disable cell membranes. Macroscopically, there appears to be some direct damage to tumor cells, extensive damage to the endothelial cells of the tumor vasculature, and infiltration of the tumor by macrophages. The macrophages remove detritus of dead cells from the PDT-treated zones of tissue, and in the process, are believed to damage living cells also.

In PDT, the porphyrins must be selectively retained by tumors, especially within the irradiation volume. However, the porphyrin drugs should be non-toxic or minimally toxic when administered in therapeutically useful doses. In addition, porphyrin drugs with absorbance peaks at long wavelengths to allow increased tissue penetration and, thereby, allow photoablation of some or all of the vasculature and/or parenchyma of deeper-seated tumors.

While it is well known in medical arts that porphyrins have been used in cancer therapy, there are several criteria that must be met for a porphyrin-mediated human cancer radiation treatment to be optimized. In BNCT, the porphyrin drug should deliver a therapeutically effective concentration of boron to the tumor while being minimally toxic to normal vital tissues and organs at a radiotherapeutically effective pharmacological whole-body dose of porphyrin. In addition, the porphyrin should have selective affinity for the tumor with respect to its affinity to surrounding normal tissues within the irradiation volume, and should be capable of achieving tumor-to-normal-tissue boron concentration ratios greater than 5:1. In vivo studies have shown that the latter criterion can be satisfied for brain tumors if the porphyrin, properly designed, synthesized and purified, does not penetrate the blood-brain-barrier in non-edematous zones of the normal CNS.

In addition, if the boron concentration and distribution in and around the tumor can be accurately and rapidly determined noninvasively, BNCT treatment planning can be more quickly, accurately, and safely accomplished. For example, neutron irradiation could be planned so that concurrent boron concentrations are at a maximum at the growing margin of the tumor rather than in the tumor as a whole. Thus, BNCT could be implemented by one relatively short exposure or a series of short exposures of mainly epithermal neutrons, appropriately timed to take advantage of optimal boron concentrations identified by SPECT or MRI in tumor, surrounding tissues, and blood in vivo. BNCT effectiveness in vivo is probably not diminished even when a neutron exposure is as short as 300 milliseconds. Such short irradiations have been delivered effectively, in fact, by a TRIGA (General Atomics) reactor operating in the pulse mode. Mice bearing advanced malignant sarcomas transplanted subcutaneously in the thigh were palliated and in many cases cured by BNCT using 300 millisecond ‘pulse’ exposures to slow neutrons (Lee E. Farr, Invited Lecture, Medical Department, Building 490, published as aBNL report around 1989-1991). Short irradiations would obviate the inconvenience and discomfort to the patient of long and often awkward positioning of the head at a reactor port. This advantage alone would justify a clinical use for BNCT, if palliative results on the tumor were at least as favorable as those following the presently, available standard, 6-week, 30-fraction postoperative linear-accelerator-based photon radiation therapy.

Efforts have been made to synthesize porphyrins for the diagnosis, imaging and treatment of cancer. In U.S. Pat. No. 4,959,356 issued to Miura, et al. (which is incorporated herein in its entirety), a particular class of porphyrins was synthesized for utilization in the treatment of brain tumors using BNCT. The porphyrins described in that patent are natural porphyrin derivatives which contain two carborane cages at the 3 and 8 positions. Natural porphyrins have particular substitution patterns which are, in general, pyrrole-substituted and asymmetric. The porphyrins described in U.S. Pat. No. 4,959,356 use heme, the iron porphyrin prosthetic group in hemoglobin, as a chemical starting material; therefore, the resulting boronated porphyrins resemble heme in their basic structure. In contrast, the porphyrins of the current invention are synthetic tetraphenylporphyrin (TPP) derivatives that are symmetrically substituted at the methine positions and most are also substituted at the pyrrole positions of the macrocycle. Acyclic precursors are used as chemical starting materials so that final product yields are generally greater than those obtained from natural porphyrin derivatives. U.S. Pat. No. 5,877,165 issued to Miura et al. (which is incorporated herein in its entirety) is focused on boronated porphyrins containing multiple carborane cages which selectivity accumulate in neoplastic tissue and which can be used in cancer therapies such as boron neutron capture and photodynamic therapy.

U.S. Pat. Nos. 5,284,831 and 5,149,801 issued to Kahl, et al. describe another type of porphyrin and their uses in BNCT, PDT and other biomedical applications. Like the porphyrins described in the previous patent by Miura et al., these are also natural porphyrin derivatives but they contain four carborane cages at the 3 and 8 positions.

U.S. Pat. No. 4,500,507 issued to Wong describes a method of labeling hematoporphyrin derivatives (HPD) with ^{99m}Tc as a means of visualizing tumors using scintigraphic noninvasive imaging techniques such as SPECT. The method taught by this patent utilizes hematoporphyrin compounds that are also natural porphyrin derivatives.

U.S. Pat. No. 4,348,376 to Goldenberg, U.S. Pat. No. 4,665,897 to Lemelson, and U.S. Pat. No. 4,824,659 to Hawthorne teach combining labeling of an antibody with ¹⁰B and with one or more other radionuclides, including those of iodine, for purposes of imaging tumors noninvasively and thereby delineating tumor targets for exposure to thermalized neutrons. Each of these patents requires that the ¹⁰B compound be linked to a radiolabeled antibody.

Improvement in the efficacy of conventional radiotherapy using chemical agents is a key area of interest in experimental radiation oncology. On an annual basis, more than 750,000 patients in the U.S. receive radiation therapy for cancer. The success has been limited due to restriction of the tumor dose to avoid critical normal tissue morbidity. Hypoxic cells in tumor can be a major problem because they are three times less sensitive to radiation than oxygenated cells. While a whole range of hypoxic cell radiation sensitizing agents have been developed, most have proven clinically ineffective. Accordingly, there is a need for effective hypoxic cell radiation sensitizing agents.

### SUMMARY OF THE INVENTION

The present invention is directed to low toxicity boronated compounds and methods for their use in the treatment, visualization, and diagnosis of tumors. More specifically, the present invention is directed to low toxicity boronated 5,10,15,20-tetraphenylporphyrin compounds and methods for their use particularly in boron neutron capture therapy (BNCT) or photodynamic therapy (PDT) for the treatment of tumors of the brain, head and neck, and surrounding tissue.

In particular, the present invention is directed to boron-containing 5, 10, 15, 20-tetraphenylporphyrins of the formula 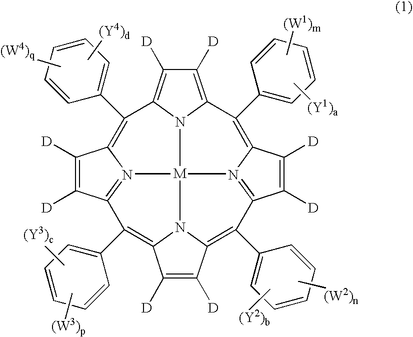
wherein: D is a halogen, a halogen isotope, a combination thereof or a combination thereof that includes from one to three hydrogen; Y¹, Y², Y³ and Y⁴ are independently on the ortho, meta or para position on the phenyl rings, and are independently hydrogen, alkyl, cycloalkyl, aryl, alkylaryl, arylalkyl, heteroaryl, or an alkyl, cycloalkyl, aryl, alkylaryl, arylalkyl, or heteroaryl group substituted with 1 to 4 hydrophilic groups selected from hydroxy, alkoxy, —C(O)OR⁵, —SOR⁶, —SO₂R⁶, nitro, amido, ureido, carbamato, —SR⁷, —NR⁸R⁹ or poly-alkyleneoxide; or a substituent represented by the following formula: —X—(CR¹R²)ᵣ-Z (formula 2), provided that at least two of (Y¹)ₐ, (Y²)_{b}, (Y³)_{c} and (Y⁴)_{d} are represented by formula (2); X is oxygen or sulfur; Z is a carborane cluster comprising at least two carbon atoms and at least three boron atoms, or at least one carbon atom and at least five boron atoms, within a cage structure; r is 0 or an integer from 1 to 20; W¹, W², W³ and W⁴ are independently hydrogen or hydrophilic groups selected from hydroxy, alkoxy, —C(O)OR⁵, —SOR⁶, —SO₂R⁶, nitro, amido, ureido, carbamato, —SR⁷, —NR⁸R⁹ or polyalkylene oxide; R¹, R², R⁵, R⁶, R⁷, R⁸ and R⁹ are independently selected from hydrogen and C₁ to C₄ alkyl; a, b, c and d independently represent an integer from 1 to 4; m, n, p and q independently represent an integer from 1 to 4, provided that at least one of m, n, p and q is not hydrogen, and each of the sums a+m, b+n, c+p and d+q, independently represents an integer from 1 to 5; and M is either two hydrogen ions; a single monovalent metal ion; two monovalent metal ions; a divalent metal ion; a trivalent metal ion; a tetravalent metal ion; a pentavalent metal ion; a hexavalent metal ion; a radioactive metal ion useful in radioisotope-mediated radiation therapy or imageable by single photon emission computed tomography (SPECT) or positron emission tomography (PET); a paramagnetic metal ion detectable by magnetic resonance imaging (MRI); a metal ion suitable for boron neutron capture therapy (BNCT) or photodynamic therapy (PDT); or a combination thereof; wherein when M is a single monovalent metal ion, the compound is charge-balanced by a counter cation; and when M is a trivalent, tetravalent, pentavalent, or hexavalent metal ion, the compound is charge-balanced by an appropriate number of counter anions, dianions, or trianions.

Preferably, Z is selected from the carboranes: —C₂HB₉H₁₀ or —C₂HB₁₀H₁₀, wherein —C₂HB₉H₁₀ is nido ortho-, meta- or para-carborane, and —C₂HB₁₀H₁₀ is closo ortho-, meta- or para-carborane and M is vanadium, manganese, iron, ruthenium, technetium, chromium, platinum, cobalt, nickel, copper, zinc, germanium, indium, tin, yttrium, gold barium, tungsten or gadolinium.

In preferred embodiments of the compound, a, b, c, and d are 1, and Y¹, Y², Y³ and Y⁴ are represented by —X—(CR¹R²)ᵣ-Z (formula 2). In other preferred embodiments, X is O; R¹ and R² are H; r is 1; and m, n, p and q are each 1. In the most preferred embodiments Y¹, Y², Y³ and Y⁴ are in the para position on the phenyl ring, and W¹, W², W³ and W⁴ are independently, hydroxy groups, which are preferably in the meta position of the phenyl ring, or alkoxy groups, preferably methoxy groups, which are preferably in the meta position of the phenyl ring. In some preferred embodiments, all of the D are halogens or halogen isotopes, most preferably bromine, iodine, a bromine isotope or an iodine isotope.

Another compound of the present invention has the formula

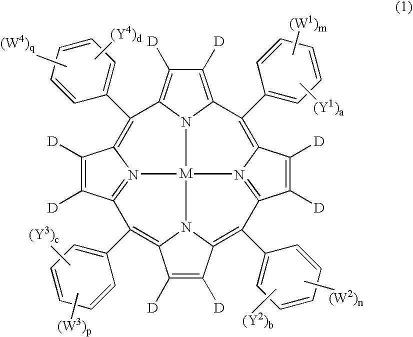

The variables are the same as previously defined, except for M, which is a trivalent, tetravalent, pentavalent or hexavalent metal ion; and wherein the porphyrin-metal complex is charge-balanced by one or more porphyrin compounds containing a divalent negative charge and represented by the formula

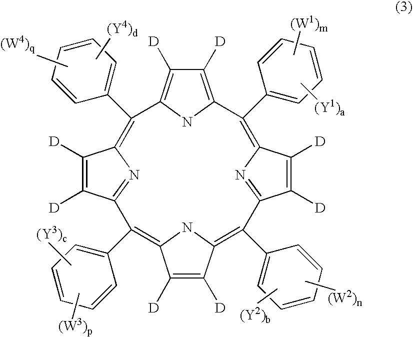

The variables for the compounds containing a divalent negative charge are the same as previously defined. These two compounds are used together and have the same methods of use as previously defined.

Another preferred compound of the present invention has the formula 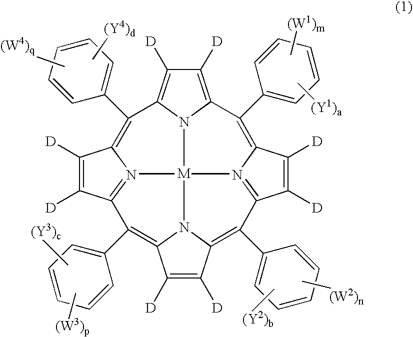
wherein: D is a halogen, a halogen isotope, a combination thereof or a combination thereof that includes from one to three hydrogen; Y¹, Y², Y³ and Y⁴ are independently on the ortho, meta or para position on the phenyl rings, and are independently hydrogen, alkyl, cycloalkyl, aryl, alkylaryl, arylalkyl, heteroaryl, or an alkyl, cycloalkyl, aryl, alkylaryl, arylalkyl, or heteroaryl group substituted with 1 to 4 hydrophilic groups selected from hydroxy, alkoxy, —C(O)OR⁵, —SOR⁶, —SO₂R⁶, nitro, amido, ureido, carbamato, —SR⁷, —NR⁸R⁹ or poly-alkyleneoxide; or a substituent represented by the following formula: —O—CH₂-Z (formula 4), provided that at least two Of (¹)ₐ, (Y²)_{b}, (Y³)_{c} and (Y⁴)_{d} are represented by formula (4); Z is a carborane cluster comprising at least two carbon atoms and at least three boron atoms, or at least one carbon atom and at least five boron atoms, within a cage structure; W¹, W², W³ and W⁴ are independently hydrogen, a hydroxyl group or an alkoxy group; R⁵, R⁶, R⁷, R⁸ and R⁹ are independently selected from hydrogen and C₁ to C₄ alkyl; a, b, c and d independently represent an integer from 1 to 2; m, n, p and q independently represent an integer from 1 to 2, provided that at least one of m, n, p and q is not hydrogen, and each of the sums a+m, b+n, c+p and d+q, independently represents an integer from 1 to 3; and M is vanadium, manganese, iron, ruthenium, technetium, chromium, platinum, cobalt, nickel, copper, zinc, germanium, indium, tin, yttrium, gold, barium, tungsten or gadolinium.

In preferred embodiments, Z is selected from the carboranes —C₂HB₉H₁₀ or —C₂HB₁₀H₁₀, wherein —C₂HB₉H₁₀ is nido ortho-, meta- or para-carborane, and —C₂HB₁₀H₁₀ is closo ortho-, meta- or para-carborane. In more preferred embodiments, a, b, c, and d are 1, m, n, p and q are each 1 and Y¹, Y², Y³ and Y⁴ are independently hydrogen or are represented by —O—CH₂-Z (formula 4). In the most preferred embodiments, Y¹, Y², Y³ and Y⁴ are in the para position on the phenyl ring, and W¹, W², W³ and W⁴ are in the meta position of the phenyl ring. In some embodiments, all of the D are halogens or halogen isotopes. Most preferably, the halogen is bromine or iodine and the halogen isotope is a bromine isotope or an iodine isotope.

The invention also includes a method of bimodal cancer treatment in a subject wherein a composition that includes one of the compounds of the present invention is administered to the subject in the vicinity of a tumor and the subject, more particularly the tumor, is then irradiated. The irradiation is preferably by a method utilizing thermal or epithermal neutrons, or laser red light. The method of bimodal cancer treatment can include boron neutron capture therapy (BNCT), X-ray radiation therapy (XRT), photodynamic therapy (PDT), single photon emission computed tomography (SPECT), positron emission tomography (PET), wherein M is a SPECT- and/or PET-imageable radioactive metal ion, or magnetic resonance imaging (MRI), wherein M is a paramagnetic metal ion.

The invention also includes a method for the of imaging a tumor and surrounding tissue in a subject, which includes administering to the subject a composition that contains a compound of the present invention and observing the metal ion in the subject, thereby imaging the tumor and surrounding tissue. The imaging preferably performed is by a method selected from magnetic resonance imaging (MRI), single photon emission computed tomography (SPECT), or positron emission tomography (PET) methods.

The present invention also provides the radiosensitizer composition according to the invention and described herein, for use in medicine. Preferably, the use is for tumor imaging and/or for a method of treating cancer. The cancer treatment may particularly be bimodal cancer treatment.

The present invention also provides the use of compounds of the invention as described herein in the manufacture of a composition for tumor imaging.

Further, the present invention provides the use of compounds of the invention as described herein in the manufacture of a composition for cancer treatment. The cancer treatment may be bimodal treatment. In such a use, the composition may well be a pharmaceutical or a medicament.

Because porphyrins used in the radiation sensitizing agents of the present invention have electron-withdrawing groups at the periphery of the macrocycle the reduction potentials are more positive than those with hydrogen or alkyl groups. Such electrochemical properties are believed to be desirable for radiosensitizers in photon radiotherapy (R. A. Miller et al., Int. J. Radiat. Oncol. Biol Phys., 45, 981-989, 1999). Coupled with their biodistribution and toxcicological properties, porphyrins of the present invention are believed to have potential as effective radiosensitizers.

### DETAILED DESCRIPTION OF THE INVENTION

The present invention relates to radiation sensitizing agents, which include halogenated (i.e., fluorinated, chlorinated, brominated and iodinated) tetracarboranylporphyrins and their use as imageable tumor-targeting agents for ionizing and/or non-ionizing radiation therapy. The halogenated tetracarboranylporphyrins are synthesized from carborane-containing tetraphenylporphyrins. The halogenated tetracarboranylporphyrins of the present invention are octahalo-analogs of the carborane-containing tetraphenylporphyrins and are prepared by synthesizing the carborane-containing tetraphenylporphyrins with a halogen in a solvent mixture such as chloroform and carbon tetrachloride.

More specifically, the present invention relates to boron-containing 5,10,15,20-tetraphenyl porphyrins having the formula 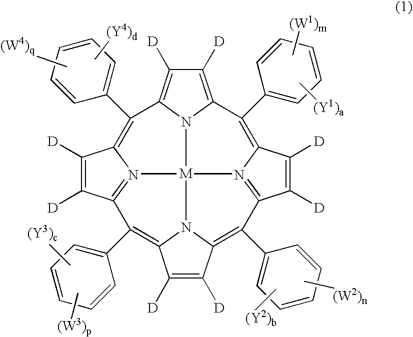
wherein D is a halogen, a halogen isotope and up to three hydrogen, preferably fluorine, a fluorine isotope, chlorine, a chlorine isotope, bromine, a bromine isotope, iodine, an iodine isotope, a combination thereof or a combination thereof that includes from one to three hydrogen. Most preferably D is bromine, a bromine isotope, iodine, an iodine isotope, a combination thereof or a combination thereof that includes one to three hydrogen.

Y¹, Y², Y³ and Y⁴, are independently on the ortho, meta or para position on the phenyl rings and Y¹, Y², Y³ and Y⁴ are independently hydrogen, alkyl, cycloalkyl, aryl, alkylaryl, arylalkyl, heteroaryl, or a substituent represented by
—X—(CR¹R²)ᵣ-Z (2)

When any of Y¹, Y², Y³ or Y⁴ is alkyl, alkyl is a straight chain or branched alkyl group containing 1 to 20 carbon atoms including, optionally, up to three double or triple bonds. Some examples of alkyl groups include methyl, ethyl, n-propyl, iso-propyl, n-butyl, iso-butyl, sec-butyl, tert-butyl, propenyl, 2-butenyl, 3-butenyl, 3-butynyl, 2-methyl-2-butenyl, n-pentyl, dodecyl, hexadecyl, octadecyl and eicosyl.

The alkyl group may be unsubstituted or substituted with 1 to 4 hydrophilic groups. Some examples of suitable hydrophilic groups include hydroxy, alkoxy, —C(O)OR⁵, —SOR⁶, —SO₂R⁶, nitro, amido, ureido, carbamato, —SR⁷, —NR⁸R⁹ and poly-alkyleneoxide. R⁵, R⁷, R⁸ and R⁹ are independently selected from hydrogen and alkyl groups as defined above, except that the alkyl groups for R⁵, R⁶, R⁷, R⁸ and R⁹ contain 1 to 4 carbon atoms.

The carbon atoms of the alkyl group may also be substituted with 1 to 4 heteroatoms. In this specification, heteroatoms are O, N, or S. The heteroatoms are not adjacent, and are separated by at least one carbon atom.

When any of Y¹, Y², Y³ or Y⁴ is cycloalkyl, the cycloalkyl ring is a 4, 5, 6, or 7 member cycloalkyl ring. The ring may be saturated, or may contain 1 to 4 unsaturated (i.e., double or triple) bonds. Some examples of saturated cycloalkyl rings include cyclobutane, cyclopentane, cyclohexane, and cyclopentane rings. Some examples of unsaturated cycloalkyl rings include cyclobutene, cyclopentene, cyclohexene, and 1,3-cycloheptadiene rings.

The cycloalkyl ring may optionally be substituted with 1 to 4 heteroatoms of O, N, or S. Some examples of cycloalkyl rings substituted with heteroatoms include pyrrolidine, piperidine, piperazine, tetrahydrofuran, furan, thiophene, 1,3-oxazolidine, imidazole, and pyrrole rings. The cycloalkyl rings may be optionally substituted with alkyl as defined above, or with 1 to 4 hydrophilic groups, also as defined above.

The cycloalkyl ring may be fused to 1 to 3 additional 4, 5, 6, or 7 member cycloalkyl or phenyl rings. Some examples of fused cycloalkyl rings are bicyclo[3.3.0]octane, bicyclo[4.3.0]non-3-ene, triphenylene and 1,2,3,4-tetrahydronaphthalene rings.

When any of Y¹, Y², Y³ or Y⁴ is aryl, aryl is a 5, 6, or 7 member aromatic ring, preferably a phenyl ring. The aryl rings may be optionally substituted with alkyl as defined above to produce alkylaryl or arylalkyl groups. The aryl, alkylaryl and arylalkyl groups may be substituted with 1 to 4 hydrophilic groups, as defined above.

The aryl ring may optionally be substituted with 1 to 4 heteroatoms of O, N, or S, resulting in a heteroaryl ring. Some examples of heteroaryl rings include thiophene, pyridine, oxazole, thiazole, oxazine and pyrazine rings. The heteroaryl ring may be substituted with 1 to 4 hydrophilic groups, as defined above.

The aryl or heteroaryl ring may also be fused to 1 to 3 additional 5, 6, or 7 member aryl or heteroaryl rings. Some examples of fused aryl and heteroaryl rings include naphthalene, anthracene, phenanthrene, triphenylene, chrysene, indoline, quinoline and tetraazanaphthalene (pteridine) rings.

At least one, and more preferably at least two, of Y¹, Y², Y³ or Y⁴ is represented by —X—(CR¹R²)ᵣ-Z, formula (2), wherein X is oxygen or sulfur, and R¹ and R² are independently selected from hydrogen and alkyl groups as defined above, except that the alkyl groups for R¹ and R² contain 1 to 4 carbon atoms. The subscript r is 0 or an integer from 1 to 20. When r is 0, the position for the Y on the phenyl ring is occupied by a hydrogen. Z is a carborane cluster that includes at least two carbon atoms and at least three boron atoms, or at least one carbon atom and at least five boron atoms, within a cage structure. Some examples of carborane clusters include the regular polyhedral carborane clusters, also known as closo structures, as well as ionized fragments of the polyhedral clusters, also known as nido structures. Some examples of the preferred carboranes of the present invention include —C₂HB₉H₁₀ or —C₂HB₁₀H₁₀, wherein —C₂HB₉H₁₀ is nido ortho-, meta-, or para-carborane, and —C₂HB₁₀H₁₀ is closo ortho-, meta-, or para-carborane.

W¹, W², W³ and W⁴ are hydrophilic groups independently selected from hydroxy, alkoxy, —C(O)OR⁵, —SOR⁶, —SO₂R⁶, nitro, amido, ureido, carbamato, —SR⁷, —NR⁸R⁹ or polyalkylene oxide, wherein R⁵, R⁶, R⁷, R⁸, and R⁹ have been previously defined.

As used herein in describing the present invention, an alkoxy group contains an alkyl portion as defined above. Some examples of alkoxy groups include methoxy, ethoxy, propoxy, n-butoxy, t-butoxy and dodecyloxy.

A polyalkylene oxide is defined according to the formula —(CH₂)_{d}—O—[(CH₂)ₑ—O—]ₓ-[(CH₂)_{f}—O—]_{y}—(CH₂)_{g}—OR′, wherein, independently, d is 0, or an integer from 1 to 10, e is 0, or an integer from 1 to 10, f is 1 to 10, g is 1 to 10, x and y are each independently 1 or 0, and R′ is either H or an alkyl group as defined previously, provided that when e is 0, then x is 0; when f is 0, then y is 0; when e is not 0, then x is 1; and when f is not 0, then y is 1.

A preferable polyalkylene oxide of the invention is polyethylene oxide. Polyethylene oxide is defined according to the formula —(CH₂)_{d}—O—[(CH₂)ₑ—O—]ₓ—[(CH₂)_{f}—]_{y}—(CH₂)_{g}—OR′, wherein, independently, d is 0 or 2, e is 0 or 2, f is 0 or 2, g is 2, x and y are each independently 1 or 0, and R′ is either hydrogen or an ethyl group, provided that when e is 0, then x is 0; when f is 0, then y is 0; when e is not 0, then x is 1; and when f is not 0, then y is 1.

The subscripts m, n, p, and q are independently 0 or an integer from 1 to 4, provided that at least one of m, n, p, and q is not zero; and the subscripts a, b, c, and d independently represent an integer from 1 to 4; provided that at least one of m, n, p, and q is not zero, and each of the sums a+m, b+n, c+p, and d+q, independently represents an integer from 1 to 5.

In formula (1), M may be two hydrogen ions, a single monovalent metal ion, or two monovalent metal ions. Some examples of suitable monovalent metal ions include Li⁺¹, Na⁺¹, K⁺¹, Cu⁺¹, Ag⁺¹, Au⁺¹ and Tl⁺¹. When M is a single monovalent metal ion, the resulting porphyrin-metal complex anion is charge-balanced by a counter cation. Some examples of counter cations include any of the foregoing monovalent metal ions, and ammonium and phosphonium cations, such as tetramethylammonium, tetrabutylammonium and tetraphenylammonium. The counter cation may be either bound or associated in some form with the porphyrin-metal complex.

M may also be a divalent metal ion. Some examples of suitable divalent metal ions include V⁺², Mn⁺², Fe⁺², Ru⁺², Co⁺², Ni⁺², Cu⁺², Pd⁺², Pt⁺², Zn⁺², Ca⁺², M⁺², S⁺² and Ba⁺².

Alternatively, M may be a trivalent, tetravalent, pentavalent or hexavalent metal ion. Some examples of suitable trivalent metal ions include Gd⁺³, Y⁺³, In⁺³, Cr⁺³, Ga⁺³, Al⁺³, Eu⁺³ and Dy⁺³. Some examples of suitable tetravalent metal ions include Tc⁺⁴, Ge⁺⁴, Sn⁺⁴ and Pt⁺⁴. An example of a suitable pentavalent metal ion is Tc⁺⁵. Some examples of suitable hexavalent metal ions include W⁺⁶, Tc⁺⁶, and Mo⁺⁶. The resulting porphyrin-metal complex cation is charge-balanced by an appropriate number of counter anions, dianions or trianions. For example, a porphyrin-metal complex cation derived from a trivalent metal ion may be charge-balanced by a single counter anion, and such a complex derived from a tetravalent metal ion may, for example, be charge-balanced by a single counter dianion or two counter anions, and so on.

Some examples of suitable counter anions include chloride, perchlorate, sulfate, nitrate and tetrafluoroborate. Some examples of suitable counter dianions include oxide, sulfide or a porphyrin compound containing a divalent negative charge. The porphyrin compound containing a divalent negative charge may be a porphyrin compound of the present invention with the proviso that M is absent. An example of a suitable counter trianion includes phosphate.

The counter anion, dianion or trianion may be either bound or associated in some form with a carborane-containing porphyrin compound of the present invention. The carborane-containing porphyrin compound may also be bound to or associated with neutrally charged molecules, such as molecules of solvation, for example, water, acetonitrile, methanol and so on.

In addition, M may be a radioactive metal ion imageable by single photon emission computed tomography (SPECT) or positron emission tomography (PET). Some examples of radioactive metals suitable for SPECT are ⁶⁷Cu, ^{99m}Tc and ¹¹¹In. Examples of radioactive metals suitable for PET include ⁶⁴Cu and ⁵⁵Co. M may also be a radioactive metal useful as a radiopharmaceutical for therapy. Some examples of radioactive metals suitable for such therapy include ⁹⁰Y, ¹⁸⁸Re and ⁶⁷Cu.

M may also be a paramagnetic metal ion detectable by magnetic resonance imaging (MRI). Some examples of such metals include Mn, Fe, Co and Gd. In addition, M may be a metal ion suitable for boron neutron capture therapy (BNCT), X-ray radiation therapy (XRT) or photodynamic therapy (PDT); or a combination thereof. The metal ions suitable for BNCT include those described thus far, with the exclusion of those that are photoactive, such as Zn and Sn. Such photoactive metals, and particularly those with long-lived triplet states, are preferable for PDT. Since the dosage for BNCT is 100 to 1000 times greater than the dosage for PDT, a significant accumulation of photoactive metal in the skin could result if such photoactive metals were used in BNCT. Such an accumulation of photoactive metal may cause biological damage.

CuOMTCPBr and CuOHTCPBr, two halogenated carborane-containing tetraphenylporphyrins, have been found to deliver high concentrations of boron to various tumors in animals with much lower amounts in normal tissues relevant for brain and head and neck cancers. It has been found that brominated porphyrins are easier to reduce than their bromine-free precursors and that they have similar biodistribution and toxicological properties. It is believed that the low reduction potential of a larger macrocycle known as gadolinium texaphyrin is largely responsible for its high in-vivo efficacy as a tumor-selective radiosensitizer during X-ray radiotherapy of tumors. However, halogenated tetracarboranylporphyrins of the present invention have a major practical advantage over the texaphyrins for BNCT because their tumor to normal brain and tumor to blood concentration ratios are 100:1 vs. 10:1 for the texaphyrins. Moreover, the porphyrins of the present invention are less toxic than Gd texaphyrin thereby allowing more drug to be administered and more compound to accumulate in tumor tissue.

The halogenated tetracarboranylporphyrins of the present invention can also be synthesized using isotopes of the different halogens. The preferred isotopes are Br-76 with a half life (T_{1/2}) of 16 hours, Br-77 (T_{1/2}=57 hours), 1-124 (T_{1/2}=101 hours), 1-131 (T_{1/2}=192 hours) and F-18 (T_{1/2}=110 minutes).

The halogenated tetracarboranylporphyrins have a range of reduction potentials that are closer to the texaphyrins than the non-brominated analogs. The examples that follow show the effectiveness of the biodistribution properties of these compounds in tests conducted using mice bearing EMT-6 carcinomas.

Photoactivation can be somewhat amplified by tuning the X-ray energy to that above the K-edge of either the metal or the halogen. The K-edge is the energy just above the binding energy of the K-shell electron, which is attracted to the nucleus of the atom and it is unique for each element. ⁶⁴Cu, ¹⁸F and ⁷⁶Br are isotopes available for quantitative positron-emission tomography (PET). The ⁶⁴Cu and ⁷⁶Br can be attached to the tetraphenylporphyrins at a late stage in the synthesis. These isotopic substitutions are expected to improve treatment planning for any future clinical applications of CuOMTCPBr or its analogs. Local concentrations of the radioactive isotope could then be visualized and quantified voxel by voxel, thereby enabling calculation of the boron concentration in the brain, head, neck or in another targeted organ or tissue of interest, voxel by voxel.

The porphyrin compounds of the present invention that have been tested in vivo are non-toxic at potentially therapeutic doses. Implementation of BNCT, XRT and/or PDT in animals and patients so dosed could selectively destroy tumor tissue without disruption of normal tissue function when irradiated with epithermal neutrons, X rays or laser light. The tumor destruction could occur without the serious side effects that may be observed in conventional tumor therapy, such as radiotherapy or chemotherapy.

To accumulate the requisite amount of a compound of the present invention in a tumor for BNCT, generally a systemically injected or infused dose of about 100-400 mg halogenated tetracarboranylporphyrin compound per kg body weight in a pharmaceutically acceptable carrier is administered to a patient. Such a carrier could include liposomes and/or commercially available solvents, such as Cremophore EL, propylene glycol, Tween 80 and the like. The compound is administered in one or more doses, the last dose being given between about one hour and one week prior to the epithermal neutron irradiation. The long retention time of any of the presently invented compounds would also permit a series of such irradiations in a so-called “fractionated irradiation schedule.” Such a schedule is deemed to be advantageous in sparing damage to normal tissues in conventional photon radiation therapy. The quantity of the halogenated tetraphenylporphyrin used in any particular treatment depends on, among other factors, the boron concentration delivered to the tumor and the toxicity of the compound at doses that are therapeutically useful.

The timing of the neutron exposure depends upon the concentration of the boron in blood, which decreases more rapidly with time than does the tumor boron concentration. The timing of the administration of the halogenated tetraphenylporphyrin depends on various considerations. Important considerations are the pharmacokinetic behavior of the compound, (e.g., the rate of absorption of the compound into the tumor and into the tumor vasculature) and the rate of excretion from and/or metabolism of the compound in the various tissues that absorb the compound in the patient.

It has long been known that porphyrins accumulate robustly in many kinds of tumors as well as in a few non-tumorous tissues. In human cancer therapy, this property has been used only for photodynamic therapy (PDT) to date. However, pre-clinical research is active in developing carboranyl derivatives of porphyrins for boron neutron-capture therapy (BNCT).

In an embodiment of the present invention, a brominated carboranylporphyrin is synthesized to provide an imageable nuclide in a porphyrin that can also be used to image a tumor non-invasively. Since the ratio of the imageable nuclide to the boron is invariant if the administered boronated compound is substantially chemically stable in vivo, quantification of the imaged nuclide, voxel by voxel, provides real-time quantification of the boron, voxel by voxel. This greatly enhances the treatment planning for clinical porphyrin-based BNCT and, therefore, adds to the potential advantage of the high tumor boron concentrations already demonstrated by some carboranyl porphyrins. An example of such a metalloporphyrins is copper octabromotetracarboranylphenylporphyrin. The bromine can be ⁷⁶Br (T_{1/2}=16 hrs), which is imageable by positron-emission tomography (PET) or ⁷⁷Br (T_{1/2}=57 hrs), which is imageable by single-photon emission computed tomography (SPECT). In another embodiment, iodine is substituted for bromine and PET and SPECT can be used with ¹²⁴I and ¹³¹I, respectively. In addition, non-radioactive natural abundance iodine can be used with spiral computed tomography (CT) to localize and quantify tumor boron rapidly by employing the iodine component of CuTCPI as a radiographic contrast-enhancing element.

The reduction potential of the porphyrin macrocycle becomes more positive (i.e., more easily reduced) with the addition of electron-withdrawing groups such as bromine. Such a physical property would be desirable in an X-ray enhancement agent. For example, the first reduction potential E_{1/2} for copper tetraphenylporphyrin (CuTPP) is −1.2 V. Whereas, that for copper octabromotetraphenylporphvrin (CuOBP) is −0.59 V. The meta-substituted carboranylmethoxy group on the phenyl moiety of copper tetracarboranylmethoxyphenylporphyrin (CuTCPH) is not expected to affect the reduction potential. Accordingly, the E_{1/2} for the octabromo derivative of CuTCPH (i.e., CuTCPBr) is estimated to be approximately −0.59 V. A similar change in the reduction potential as the macrocycle becomes more positive exists for halogenated tetracarboranylporphyrins.

The radiation-enhancement properties of gadolinium texaphyrins are attributed to their relatively large reduction potentials, −0.04 V. However, reduction potentials that are optimal for radiotherapy have not yet been determined. The eight bromo groups in CuOHTCPBr provide moderately strong electron-withdrawing groups to the tetraphenylporphyrin structure. If more positive reduction potentials are required for greater efficacy in the control of neoplastic tissues, groups with greater electron-withdrawing properties such as fluoro or nitro groups can be used in place of the bromo substituents.

Tests in animals have shown that the halogenated tetracarboranylporphyrins of the present invention provide low toxicity and high tumor accumulation of the described porphyrins. In addition, the halogenated tetracarboranylporphyrins of the present invention can be used in a variety of cancer treatment modalities and they are imageable by a number of different methods.

The invention also relates to methods of treating tumors. In a preferred embodiment, the method of treating malignant tumors, especially brain tumors, is via BNCT. BNCT is a bimodal cancer treatment based on the selective accumulation of a stable nuclide of boron known as boron-10, or ¹⁰B, in the tumor, followed by irradiation of the tumor with thermalized neutrons. The thermalized neutrons impinge on the boron-10, causing a nuclear fission reaction. The nuclear fission causes the highly localized release of vast amounts of energy in the form of high linear-energy-transfer (LET) radiation, which can more effectively kill cells than low LET radiation, such as x-rays.

Boron-10 undergoes the following nuclear reaction when captured by a thermal neutron:
¹⁰B+n→¹¹B
¹¹B→⁷Li+⁴He+γ(478 keV)

In this nuclear reaction, a boron-10 nucleus captures a neutron forming the metastable nuclide ¹¹B, which spontaneously and nearly instantaneously disintegrates into a ⁴He and ⁷Li particle, which together possess an average total kinetic energy of 2.34 MeV. These two ionized particles travel about 9 μm and 5 μm (7±2 μm) in opposite directions in soft tissue, respectively.

The distances traveled by the ⁴He and ⁷Li particles are comparable to the diameter of many tumor and tumor-associated cells. Therefore, the efficacy of BNCT resides in the production of highly localized, high LET ionizing radiation within the tumor. The targeted tumor thus receives a large dose of radiation while sparing surrounding normal tissue.

In the case of brain tumors, after administration of the halogenated carborane-containing tetraphenylporphyrin compound, the patient's head is irradiated in the general area of the brain tumor with an incident beam or field of epithermal (0.5 eV-10 keV) neutrons. The neutrons become progressively thermalized (average energy approximately 0.04 eV) as they penetrate deeper into the head. As the neutrons become thermalized, they are more readily captured by the boron-10 concentrated in the tumor cells and/or tumor supporting tissues, since the capture cross section is inversely proportional to the neutron velocity.

In BNCT of malignant brain tumors following the method of the present invention, the patient is first given an infusion of a halogenated carborane-containing tetraphenylporphyrin of formula (1), which is highly enriched in boron-10. The halogenated carborane-containing tetraphenylporphyrin is then concentrated, preferentially in the brain tumor, within the effective irradiation volume, which, for brain tumors, may be a substantial part of the brain. For example, tumors located in most or all of one hemisphere and some or all of the contralateral hemisphere of the brain can accumulate boronated porphyrins.

The tumor area is then irradiated with thermalized neutrons (primary irradiation), some of which are captured by the boron-10 concentrated in the tumor. The relative probability that the slow-moving thermal neutrons will be captured by the boron-10 nuclide is high compared to the probability of capture by all of the other nuclides normally present in mammalian tissues, provided that boron-10 concentrations in tumor tissues is greater than 30 μg/g.

Since a minuscule proportion of the boron-10 nuclei in and around a tumor undergoes the nuclear reaction immediately after capturing a neutron, a high concentration of boron-10 in the targeted tissue is necessary for BNCT to be clinically effective. Therefore, to maximize the concentration of boron-10 in the targeted tissue, the carborane clusters are highly enriched in boron-10. Specifically, the boron in the carborane cluster is enriched to at least 95 atom % in boron-10.

An advantage of the present invention over the prior art for the treatment of cancer is that the halogenated carborane-containing tetraphenylporphyrins of the present invention selectively accumulate in neoplasms in more preferred ratios than other known boron-containing compounds

Additionally, the porphyrin compounds of the present invention that have been tested in vivo are non-toxic at theoretically therapeutic effective doses. The higher selectivity and lower toxicity of the halogenated carborane-containing tetraphenylporphyrins of the present invention allow for the selective destruction of tumor tissue with minimal disruption of normal tissues and tissue function when irradiated.

Another advantage of the halogenated carborane-containing tetraphenylporphyrins of the present invention is their increased polarity, imparted through polar groups W¹, W², W³ and W⁴ on the phenyl rings. The greater polarity of such groups render the tetraphenylporphyrin compounds less lipophilic, which effects a reduction of the amount of an emulsifying co-solvent during administration. Therefore, the microlocalization within the tumor cell may be improved yielding a higher relative biological effect.

In addition, the ether linkages in the halogenated carborane-containing tetraphenylporphyrins of the present invention are more polar (particularly CuOHTCPBr) than its precursors and, therefore, provide a further reduction in lipophilicity. At the same time, the ether linkages possess greater resistance to hydrolysis than other linkages such esters and amides.

It is significant that the halogenated carborane-containing tetraphenylporphyrins of the present invention may contain in excess of 8 carborane clusters (80 boron atoms). In fact, the present invention includes carborane-containing porphyrin molecules containing 16 carborane clusters, which is higher than any carborane-containing porphyrin currently known. Since such high carborane-containing porphyrin molecules deliver more boron to a target, i.e., they are more potent, they permit lower required molar doses of porphyrin as compared to the porphyrin compounds in the prior art. The lower molar dose of halogenated carborane-containing tetraphenylporphyrin allows the amount of boron at the target to be significantly increased while keeping blood porphyrin concentrations well below toxic threshold values.

To accumulate the requisite amount of a compound of the present invention in a tumor, generally a systemically injected or infused dose of about 10-50 milligrams of boron-10 per kg body weight in a pharmaceutically acceptable carrier is administered to a patient. The carrier may include such commercially available excipients as Cremophor EL, propylene glycol, Tween 80, polyethylene glycol, ethanol, or liposomes. The compound is administered in one or more doses, the last dose being given between about 1 hour and one week prior to the epithermal neutron irradiation.

The timing of the neutron exposure depends upon the concentration of the porphyrin in the blood, which decreases more rapidly with time than the porphyrin concentration in the tumor. However, the timing of the administration of the halogenated carborane-containing tetraphenylporphyrin depends on various considerations that are well known to those skilled in the art of clinical BNCT, including the pharmacokinetic behavior of the compound, (e.g., the rate of absorption of the compound into the tumor and into the tumor vasculature) and the rate of excretion from and/or metabolism of the compound in the tumor and various other tissues that absorb the compound.

In another preferred embodiment of the present invention, the method of treating malignant tumors is via XRT. XRT is typically conventional radiotherapy but additionally involves the administration of a radiation enhancement agent such as nitroimidazole or Gd texaphyrin to the patient prior to irradiation. XRT can also be a radiosurgical modality such as gamma knife or intensity modulated radiation therapy [IMRT], which generally requires fewer fractions than conventional XRT.

In another preferred embodiment of the present invention, the method of treating malignant tumors is via PDT. PDT is a bimodal cancer treatment based on the selective accumulation of a porphyrin in a tumor, followed by irradiation of the tumor with laser red light. Upon activation with light, an electron of the porphyrin is excited from the singlet ground state to a singlet excited state. The electron then can either return to the singlet ground state with the emission of light causing fluorescence, or it can change its spin via intersystem crossing to the triplet state. In the decay of the triplet back down to the ground state singlet, it can transfer energy to ground state triplet dioxygen which forms the highly reactive singlet oxygen. Biomolecules that react most readily with singlet oxygen include unsaturated lipids and alpha amino-acid residues, both of which are major constituents of biological membranes. Beyond a certain reversible or repairable threshold, damage to membranes, especially to endothelial cell membranes, can lead to local vascular thrombosis and shutdown of blood circulation.

In using PDT in the present invention, the patient is first given an injection or infusion of a photosensitizing halogenated carborane-containing tetraphenylporphyrin of formula (1). Fiber-optic probes are then used to illuminate the tumor tissue. For malignant tumors, it is preferable that the PDT photosensitizers have optical absorbance peaks at sufficiently long wavelengths for maximum penetration to the depth of the tumor.

In a preferred embodiment, the therapeutic treatment of malignant tumors is augmented by the use of SPECT or PET. In SPECT, the patient is first given an infusion or injection of a compound of formula (1) wherein M is a gamma-emitting radioactive metal ion. The patient's head is then scanned noninvasively and the radionuclide concentration, and hence indirectly, the average boron concentration, in each pixel or voxel representing brain or brain tumor tissue is imaged. Contour lines representing zones of equal boron-10 concentration can thereby be drawn on each image of the brain.

SPECT of the brain is at least one order of magnitude more sensitive to isotopic tracers than is conventional radiography or computerized tomography. In addition, SPECT results, as opposed to results from conventional radiography, can be analyzed to provide quantitative information either in defined volumes or voxels of the brain images, in the concentrations of boron relevant to BNCT treatment planning and implementation. SPECT scanning can indicate the presence of a tumor in the patient, as well as its location in the brain or elsewhere in the body. SPECT scanning is noninvasive, fast, and convenient.

However, the positron emitting PET-imageable radioisotope Cu-64, is more readily available than is Cu-67, used in SPECT. Because of the much greater availability of Cu-64, we have carried out preclinical PET studies using a Cu-64 labeled porphyrin.

In another preferred embodiment, the therapeutic treatment of malignant tumors is augmented by the use of MRI. In MRI, a patient is first given an infusion or injection of a solution containing a halogenated carborane-containing tetraphenylporphyrin of formula (1) chelated to a suitable paramagnetic metal ion. For a brain tumor, the patient's head is then scanned and the paramagnetic metal ion concentration, and thus, boron concentration in the brain is imaged and quantified. MRI utilizing the compounds of the present invention may permit rapid enhanced targeting and treatment planning for neutron irradiation in BNCT before, during and after infusion when the boronated compound is being redistributed in blood, tumor, and healthy tissue.

The halogenated carborane-containing tetraphenylporphyrins of the present invention are synthesized through a series of separate steps. Provided below is first, a summary of the synthetic steps required for the preparation of the preferred halogenated carborane-containing tetraphenylporphyrins of the present invention, wherein Y¹, Y², Y³ and Y⁴ are represented by the formula —X—(CR¹R²)ᵣ-Z, formula (2). The synthetic summary provides general methods for synthesizing compounds of the invention and, thereby, includes several different specific ways to achieve any one synthesis. For example, different starting materials may be used to synthesize the same product, and each starting material may require a different set of reaction conditions such as temperature, reaction time, solvents and extraction and purification procedures.

The specific examples describe a preferred method for synthesizing the halogenated carborane-containing tetraphenylporphyrin compounds of the present invention. The scope of this invention is not to be in any way limited by the examples set forth herein. For example, assymetric carborane-containing tetraphenylporphyrin compounds can be synthesized by using a mixture of different benzaldehyde or dibenzaldehyde starting materials and proceeding with a similar synthetic reaction as shown in reaction scheme 6.

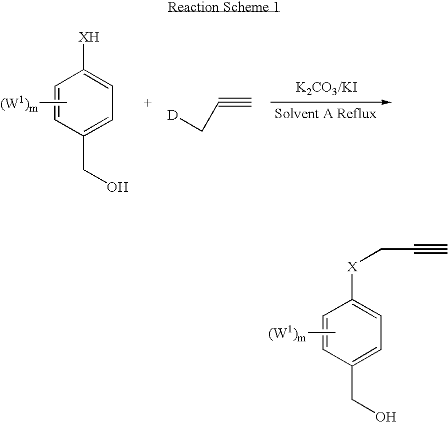

For this reaction, X is either O or S, D is a halogen, solvent A is preferably a polar non-protic solvent such as acetone; W¹ is hydrogen, hydroxy, alkoxy, —C(O)OR⁵, —SOR⁶, —SO₂R⁶, nitro, amido, ureido, carbamato, —SR⁷, —NR⁸R⁹, poly-alkyleneoxide, wherein R⁵, R⁶, R⁷, R⁸ and R⁹ are independently selected from hydrogen and C₁ to C₄ alkyl; and m is 0 or an integer from 1 to 4.

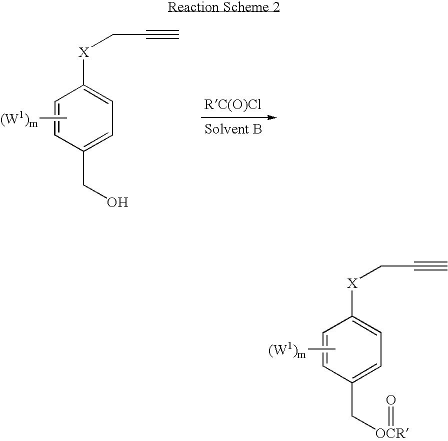

For this reaction, X, W¹ and m are as defined above, solvent B is preferably a proton scavenger such as pyridine, and R′ is an alkyl, cycloalkyl or aryl group.

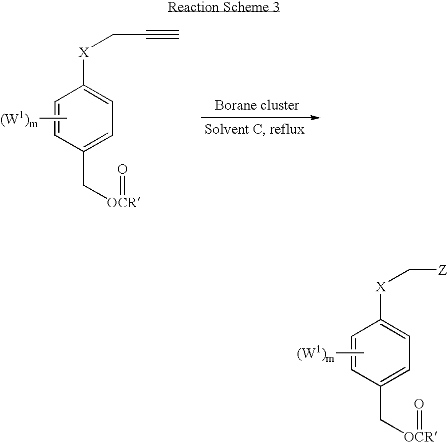

For this reaction, X, W¹, m and R′ are as defined previously, and solvent C is preferably a higher boiling hydrocarbon such as toluene. The borane cluster is any cluster comprising at least three boron atoms, or at least one carbon atom and at least five boron atoms, within a cage structure. For example, the borane cluster can be decaborane, B₁₀H₁₄. The borane cluster reacts with the triple bond of the propargyl starting material to form the carboranyl product. Thus, in the case of decaborane, Z represents the carborane —C₂HB₁₀H₁₀. Z represents any carborane cluster comprising at least two carbon atoms and at least three boron atoms, or at least one carbon atom and at least five boron atoms, within a cage structure. For example, the carborane cluster may be —C₂HB₉H₁₀ or —C₂HB₁₀H₁₀, wherein —C₂HB₉H₁₀ is nido ortho-, meta-, or para-carborane, and —C₂HB₁₀H₁₀ is closo ortho-, meta-, or para-carborane.

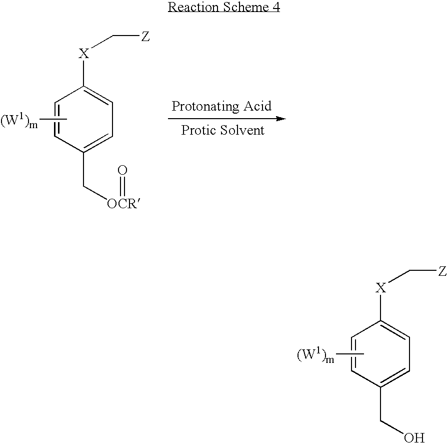

For this reaction, X, W¹, m, R′, and Z are as defined previously. The protonating acid is any acid, acid mixture, or sequence of acid additions capable of converting the ester into the alcohol product. Preferably, the protonating acid is concentrated HCl. The protic solvent may be, for example, an alcohol such as methanol.

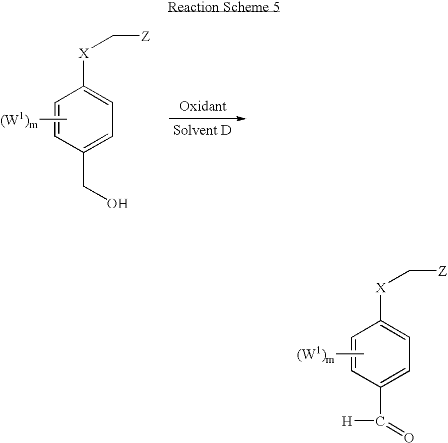

For this reaction, X, W¹, m and Z are as defined previously, solvent D is a polar non-protic solvent, preferably dichloromethane, and the oxidant is any oxidizing compound capable of selectively converting a primary alcohol to an aldehyde, preferably 2,3-dichloro-5,6-dicyano-1,4-benzoquinone (DDQ) or pyridinium chlorochromate (PCC).

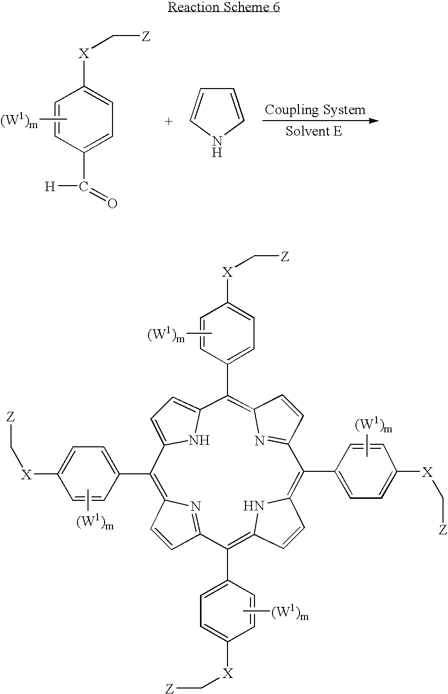

For this reaction, X, W¹, m and Z are as defined previously. The coupling system preferably comprises a Lewis acid (electron acceptor) such as boron trifluoride (BF₃) or trifluoroacetic acid (TFA) to form the intermediate porphyrinogen from the pyrrole and benzaldehyde and an oxidizing agent such as 2,3-dichloro-5,6-dicyano-1,4-benzoquinone (DDQ) to oxidize the porphyrinogen to porphyrin. Solvent E is a nonpolar non-protic solvent, preferably dichloromethane.

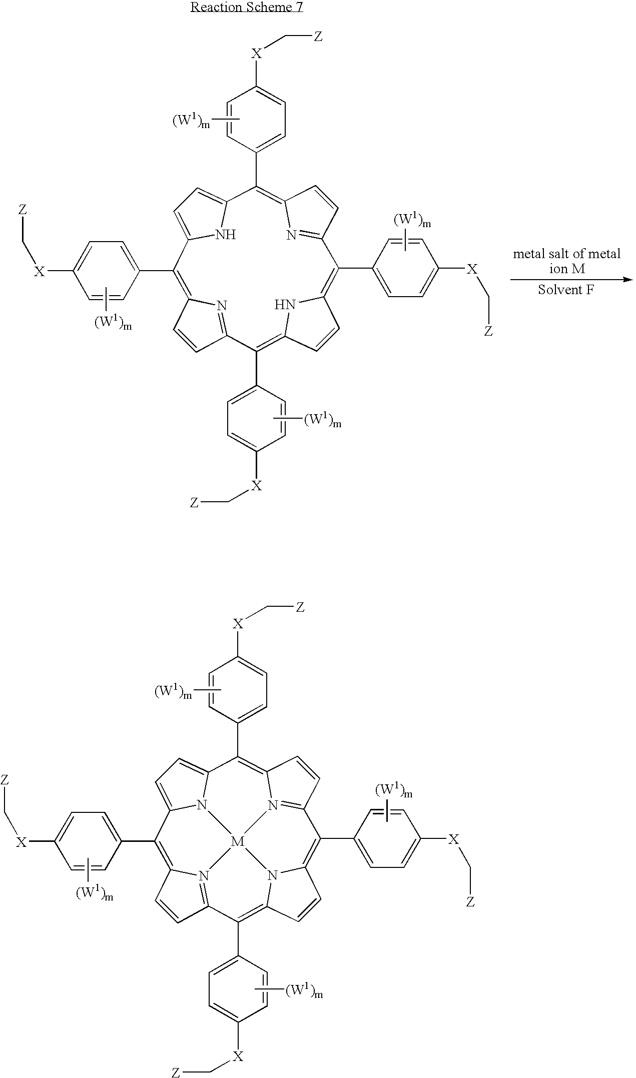

For this reaction, X, W¹, m and Z are as defined previously. In a preferred embodiment, M is selected from the group consisting of vanadium (V), manganese ruthenium (Ru), technetium (Tc), chromium (Cr), platinum (Pt), cobalt (Co), nickel (Ni), copper (Cu), zinc (Zn), germanium (Ge), indium (In), tin (Sn), yttrium (Y), gold (Au), barium (Ba), tungsten (W) and gadolinium (Gd). In a more preferred embodiment, M is copper (Cu) or nickel (Ni). The metal salt used contains the metal ion M chelated to the porphyrin. For example, for the compound where M is desired to be copper, copper acetate, i.e., Cu(OAc)₂H₂O, may be used as the metal salt. Solvent F is any solvent or solvent mixture capable of at least partially solubilizing the porphyrin and metal salt, and that does not interfere with incorporating the metal into the porphyrin.

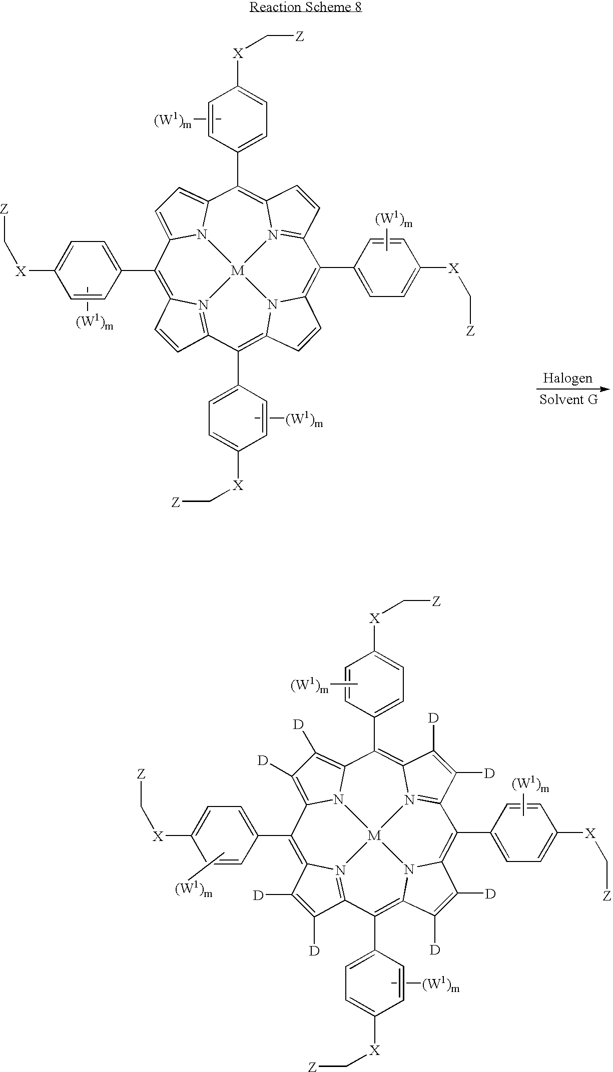

For this reaction, X, W¹, m, M and Z are as defined previously. D is selected from the group consisting of fluorine, a fluorine isotope, chlorine, a chlorine isotope, bromine, a bromine isotope, iodine, an iodine isotope, a combination thereof or a combination thereof that includes from one to four hydrogens. For example, the halogenating agent can be D₂, such as Br₂, in the specific case of bromine. More typically, the halogenating agent can be N-bromosuccinimide (NBS) for bromine or for the chlorine analog, NCS. Solvent G is any solvent or solvent mixture capable of at least partially solubilizing the porphyrin and the halogenating agent. Example as of such solvents are chloroform, carbon tetrachloride, dichloromethane, and methanol.

### EXAMPLES

Examples have been set forth below for the purpose of illustration and to describe the best mode of the invention at the present time. The scope of the invention is not to be in any way limited by the examples set forth herein.

For the syntheses carried out in the following examples, all reagents were purchased from Aldrich Chemical Co., Milwaukee, Wis., unless otherwise stated and used without further purification. Silica gel (Aldrich—200-400 mesh) was used for column chromatography. Analytical thin layer chromatography (TLC) plates were Baker-flex F254 silica gel (precoated sheets, 2.5×7.5 cm). Reactions were monitored by TLC and by optical absorption spectroscopy (Cary 50 CONC UV-Visible spectrophotometer, Varian Inc., Palo Alto, Calif.).

The high pressure liquid chromatograph (HPLC) system that was used included a HPLC Column from Phenomenex Prodigy™, ODS(3), 5μ, 100 [symbol] , 150 mm×4.6 mm, 5 μm particle size. The chromatograph was Hewlett Packard Model HP 1050 using LC ChemStation, revision A.06.04, software. A diode array detector was used with a wavelength of 463 nm and a 4 nm bandwidth (reference: 599 nm, 2 nm bandwidth). The solvent flow rate was 1 mL/min using a solvent system of acetonitrile/methanol gradient 50:50 to 0:100 in 15-35 minimum back to 50:50 in 40 minimum with a sample volume of 20 μL.

### Example 1

### Synthesis of 3-methoxy-4-propargyloxybenzylalcohol (I)

Finely powdered K₂CO₃, 10.4 grams (0.075 moles), and KI, 9.1 grams (0.060 moles), were placed in a 300 mL round-bottomed flask equipped with a magnetic stir bar, and 150 mL acetone was added. 3-methoxy-4-hydroxybenzyl alcohol, 7.71 grams (0.050 moles), and propargyl chloride, 4.10 grams (0.055 moles), were then added, and the mixture stirred and refluxed for approximately 48 hours. The results from thin layer chromatography showed no starting material (3-methoxy-4-hydroxybenzyl alcohol) as well as the presence of a new compound. The solution was then filtered. The acetone of the resulting filtrate was removed by rotary evaporation, leaving an oily residue. The oily residue was dissolved in 50 mL dichloromethane and washed with water (30 mL×2) and then dried over anhydrous potassium carbonate. After filtering the organic phase, the solvents were removed by rotary evaporation, leaving a liquid product. 9 grams of product was obtained, which corresponds to a 94% yield.

The product gave the following proton nuclear magnetic resonance (¹H NMR) spectrum in ppm (in CDCl₃ solvent): 2.49 (triplet, 1H, alkynyl); 2.57 (singlet, 1H, hydroxyl); 3.81 (singlet, 3H, methyl); 4.55 (doublet, 2H, methylene); 6.83 (multiplet, 1H, aryl); 6.89 (multiplet, 1H, aryl); 6.94 (multiplet, 1H, aryl). The product gave the following proton-decoupled carbon-13 nuclear magnetic resonance (¹³C NMR) spectrum in ppm (in CDCl₃ solvent): 55.8 (methylene); 56.8 (methyl); 64.8 (methylene); 75.8 (alkynyl); 78.5 (alkynyl); 110.2 (aryl); 114.3 (aryl); 119.0 (aryl); 135.2 (aryl); 146.0 (aryl); 149.7 (aryl). The mass spectrum (FAB) showed a parent ion peak of 192.1 that matched the molecular weight of the compound.

### Example 2

### Synthesis of 3-methoxy-4-propargyloxybenzyl acetate (II)

Acetyl chloride, 1.38 grams (0.0176 moles), was dissolved in 10 mL of pyridine in a 100 mL round flask cooled in an ice bath. A solution of 3-methoxy-4-propargyloxybenzylalcohol (I), made by dissolving 2.82 grams (0.0146 moles) of (I) in 15 mL pyridine, was added dropwise into the flask. The mixture was stirred for five hours, after which time the solvent was removed by rotary evaporation. The resulting residue was cooled to room temperature, and then dissolved in dichloromethane (30 mL). The organic phase was washed with aqueous 3N HCl and then water and dried over anhydrous magnesium sulfate. After filtering, the solvent of the organic phase was removed by rotary evaporation, leaving a yellow oil, which solidified upon standing. Recrystallization in methanol yielded 2.91 grams of the white crystalline solid, which corresponds to an 85% yield.

The product had a melting point of 69-71° C. and gave the following ¹H NMR spectrum in ppm (in CDCl₃ solvent): 2.09 (singlet, 3H, methyl); 2.50 (triplet, 1H, alkynyl); 3.89 (singlet, 3H, methyl); 4.76 (doublet, 2H, methylene); 5.05 (singlet, 2H, methylene); 6.92 (singlet, 1H, aryl); 6.93 (multiplet, 1H, aryl); 7.01 (doublet, 1H, aryl). The product gave the following proton-decoupled carbon-13 nuclear magnetic resonance (¹³C NMR) spectrum in ppm (in CDCl₃ solvent): 21.2 (methyl); 56.1 (methyl); 56.9 (methylene); 66.6 (methylene); 76.0 (alkynyl); 78.6 (alkynyl); 112.4 (aryl); 114.3 (aryl); 121.1 (aryl); 130.0 (aryl); 147.0 (aryl); 149.8 (aryl); 171.0 (carbonyl). The mass spectrum (FAB) showed a parent ion peak of 234.6 that matched the molecular weight of the compound.

### Example 3

### Synthesis of 3-methoxy-4-o-oxymethylcarboranylbenzyl acetate (III)

Decaborane, 2.07 grams (0.017 moles), was stirred in 100 mL of toluene in a 250 mL round-bottomed flask at room temperature under an argon atmosphere. Acetonitrile, 2.1 mL (0.040 moles), was added by syringe. The mixture was allowed to stir for three hours. 3-methoxy-4-propargyloxybenzyl acetate (II), 3.82 grams (0.0163 moles), was then added, and the mixture slowly heated to 80-90° C. The mixture was maintained at a temperature of 80-90° C. under an argon atmosphere for three days, after which time the results from thin layer chromatography showed the no presence of starting material (II) as well as the presence of a new compound. The solvents from the mixture were then removed by rotary evaporation. The resulting residue was dissolved in 50 mL of dichloromethane, which was washed with 20 mL of 10% sodium bicarbonate and then twice with water (20 mL each), and then dried over anhydrous sodium sulfate. After filtering the organic phase, the solvent was removed by rotary evaporation, leaving a yellow oil which crystallized upon standing. 4.64 grams of product was obtained, which corresponds to an 80% yield.

The product had a melting point of 84-85° C. and gave the ¹H NMR spectrum in ppm (in CDCl₃ solvent): 2.00 (singlet, 3H, CH₃); 3.76 (singlet, 3H, OCH₃); 4.29 (singlet, 1H, CH); 4.54 (singlet, 2H, CH₂CCHB₁₀H₁₀); 4.95 (singlet, 2H, ArCH₂); 6.74 (multiplet, 2H, ArH); 7.17 (singlet, 1H, ArH). The product gave the following proton-decoupled ¹³C NMR spectrum in ppm (in CDCl₃ solvent): 21.1 (OCH₃); 56.0 (ArOCH₂); 58.0 (OCH₃); 66.4 (ArCH₂); 71.6 (—CCHB₁₀H₁₀); 72.1 (—CCHB₁₀H₁₀); 112.8 (aryl); 116.8 (aryl); 121.2 (aryl); 132.0 (aryl); 146.8 (aryl); 150.4 (aryl); 171.0 (CO). The mass spectrum (FAB) showed a parent ion peak of 352.8 that matched the molecular weight of the compound.

### Example 4

Synthesis of 3-methoxy-4-o-oxymethylcarboranylbenzyl alcohol (IV)

Concentrated hydrochloric acid, 2 mL, was added to a solution composed of 4 grams (11 millimoles) of 3-methoxy-4-o-oxymethylcarboranylbenzyl acetate (III) in 50 mL methanol. The mixture was refluxed for three hours, after which time the results from thin layer chromatography showed no presence of starting material (III) and the presence of a new compound. The solvents were then removed by rotary evaporation, leaving a gold-colored oil. On standing at room temperature, the oil solidified to a semisolid. 3.50 grams of product was obtained, which corresponds to a 99% yield.

The product gave the following proton nuclear magnetic resonance (¹H NMR) spectrum in ppm (in CDCl₃ solvent): 3.39 (singlet, 3H, OCH₃); 3.85 (singlet, 2H, ArCH₂); 4.33 (singlet, 1H, CH); 4.39 (singlet, 2H, CH₂CCHB₁₀H₁₀); 6.85 (multiplet, 2H, ArH); 6.92 (multiplet, 1H, ArH). The product gave the following proton-decoupled ¹³C NMR spectrum in ppm (in CDCl₃ solvent): 55.9 (ArOCH₃); 58.0 (OCH₃); 58.3 (ArCH₂); 71.7 (—CCHB₁₀H₁₀); 74.4 (—CCHB₁₀H₁₀); 112.0 (aryl); 117.0 (aryl); 120.3 (aryl); 134.5 (aryl); 146.4 (aryl); 150.5 (aryl).

### Example 5

### Synthesis of 3-methoxy-4-o-oxymethylcarboranylbenzaldehyde (V)

Method 1: Pyridinium chlorochromate (PCC), 2.3 grams (11 millimoles), was stirred in 25 mL dichloromethane in a flask submerged in an ice bath. A solution of the 1.71 grams (5.5 millimoles) 3-methoxy-4-o-oxymethylcarboranyl benzyl alcohol (IV) dissolved in 25 mL dichloromethane was added dropwise to the cooled PCC solution. The resulting mixture was stirred for two hours, after which time thin layer chromatography showed no presence of starting material (IV) as well as the presence of a new compound. The resulting black heterogeneous solution was filtered through a sintered glass funnel containing silica (2 cm). The silica was washed thoroughly with additional dichloromethane to extract the product. The solvents were removed from the filtrate by rotary evaporation, leaving an oily residue, which solidified upon standing. 1.6 grams of product was obtained, which corresponds to a 94% yield.

Method 2: Equimolar amounts of (IV) and 2,3-dichloro-5,6-dicyano-1,4-benzoquinone (DDQ) were stirred in dioxane for 1 hour. The solvent was then removed by rotary evaporation. Dichloromethane was then added to selectively extract the product. The insoluble DDQH₂ side-product was removed by filtration. Rotary evaporation of the resulting filtrate yielded the final product.

The product had a melting point of 146-147° C. and gave the following ¹H NMR spectrum in ppm (in CDCl₃ solvent): 3.92 (singlet, 3H, OCH₃); 4.28 (singlet, 1H, CH); 4.51 (singlet, 2H, CH₂CCHB₁₀H₁₀); 6.92 (singlet, 1H, ArH); 7.44 (multiplet, 2H, ArH); 9.88 (singlet, 1H, CHO). The product gave the following proton-decoupled carbon-13 nuclear magnetic resonance (¹³C NMR) spectrum in ppm (in CDCl₃ solvent): 56.2 (ArOCH₂); 58.1 (OCH₃); 70.6 (—CCHB₁₀H₁₀); 71.4 (—CCHB₁₀H₁₀); 110.3 (aryl); 114.4 (aryl); 126.0 (aryl); 132.3 (aryl); 150.6 (aryl); 190.9 (CO). The mass spectrum (FAB) showed a parent ion peak of 309.7 that matched the molecular weight of the compound.

### Example 6

### Synthesis of meso-5,10,15,20-tetrakis [3-methoxy-4-o-oxymethylcarboranylphenyl]porphyrin (VI)

3-methoxy-4-o-oxymethylcarboranylbenzaldehyde (V), 50 milligrams (0.136 millimoles), was placed in a dry 100 mL round-bottomed flask and stoppered with a rubber septum. A solution of freshly distilled pyrrole, 9.5 microliters (0.136 millimoles) of pyrrole in 40 mL of dichloromethane, was transferred by syringe to the flask containing (V). The resulting mixture was deoxygenated by bubbling argon directly into the solution (with an outlet needle in septum) while stirring for 15 to 20 minutes. Trifluoroacetic acid (TFA), 5.4 microliters (0.045 millimoles), was added to the mixture using a microliter syringe. The mixture was allowed to stir under an argon atmosphere overnight. DDQ, 34 milligrams (0.149 millimoles), was then added, which immediately turned the solution very dark. The solution was refluxed for one hour. The solution was then purified using a 30 mL sintered glass funnel containing about 20 mL silica. The resulting dark filtrate was rotary evaporated to dryness. The results from thin layer chromatography confirmed the presence of the porphyrin product as well as some contaminants. The solid was redissolved in dichloromethane and then further purified using another short column of silica eluting with a 1:1 solvent mixture of dichloromethane to hexanes. The results from thin layer chromatography confirmed the absence of the contaminants. The resulting dark filtrate was rotary evaporated to dryness, resulting in 15 milligram, of product, which corresponds to a 31% yield.

The product gave the following proton nuclear magnetic resonance (¹H NMR) spectrum in ppm (in CDCl₃ solvent): −2.77 (singlet, 2H, NH); 3.94 (singlet, 2H, OCH₃); 4.50 (singlet, 4H, CH); 4.74 (singlet, 8H, CH₂CCHB₁₀H₁₀); 7.21 (doublet, 4H, ArH); 7.72 (doublet, 4H, ArH); 7.77 (singlet, 4H, ArH); 8.85 (singlet, 8H, pyrrole-H). The mass spectrum (FAB) showed a parent ion peak of 1424.7 that matched the molecular weight of the compound. The ultraviolet-visible absorbance spectrum of the product in dichloromethane showed the following peaks in nanometers of wavelength: 423, 517, 554, 593, and 648.

### Example 7

### Synthesis of copper meso-5,10,15,20-tetrakis [3-methoxy-4-o-oxymethylcarboranyl phenyl]porphyrin (VII)

A solution of Cu(OAc)₂H₂O (20 milligrams, 100 millimoles) in 5 mL methanol was added into a solution of porphyrin compound (VI) (130 milligrams, 91 millimoles) in 10 mL dichloromethane. The mixture was stirred for 20 minutes. The solvent was then removed by rotary evaporation. The resulting residue was dissolved in dichloromethane, washed with water and then dried over anhydrous sodium sulfate. The drying agent was filtered off. The solvent of the filtrate was removed by rotary evaporation, leaving a red solid residue. The solid was re-dissolved in dichloromethane and purified using a silica pad eluting with a 1:1 solvent mixture of hexane and dichloromethane. The solvents were removed by rotary evaporation, leaving the red copper porphyrin compound, 132 milligrams, which corresponds to 98% yield.

The mass spectrum (FAB) showed a parent ion peak of 1486.3 that matched the molecular weight of the compound. The ultraviolet-visible absorbance spectrum of the product showed the following peaks in nanometers of wavelength (in dichloromethane solvent): 418, 542.

### Example 8

### Synthesis of CuOMTCPBr (copper meso-5,10,15,20-tetrakis [3-methoxy-4-o-oxymethylmethoxycarboranyl phenyl]octabromoporphyrin) (VIII)

In this example, 20 mg, 0.0134 mmol of CuOMTCPH (porphyrin compound (VII) which was synthesized in Example 7) was dissolved in 3 mL of anhydrous chloroform (CHCl₃) in a clean, dry round-bottom flask equipped with a magnetic stir bar. To this mixture, 26.3 mg, 0.148 mmol of N-bromosuccinimide (C₄H₄BrNO₂ or NBS) dissolved in 2 mL of anhydrous methanol was added. The reaction was allowed to reflux under nitrogen overnight. After 18 hours reflux, the optical spectra of the mixture showed no starting material (418, 542 nm) but showed a broad peak with λₘₐₓ at 450 nm and HPLC showed 3 peaks (27.9 min, 14.4%; 32.3 min, 60.1%; 38.5 min 24.7%). Another 10 molar equivalents of NBS were added. After stirring at reflux overnight, the HPLC measurements showed a major peak (86%) at a 28.4 minute-retention time and a λₘₐₓ of 471 nm. At this time, another mole equivalent of NBS was added, but after 3 more hours there was no change in HPLC, so the reaction was quenched with an aqueous saturated sodium metabisulfite solution and then worked up. Work up was carried out by diluting the reaction mixture with dichloromethane [DCM] and then washing the organic layer three times with water, drying it over anhydrous sodium sulfate and removing the solvents by rotary evaporation.

The product was purified by preparative TLC (1 mm silica, 50% hexane/DCM). HPLC of this product showed 89.2% purity. When the reaction was carried out in a similar manner but over 7 days, a comparable yield of 90.6% yield was obtained. The red-shifted Soret band at 471 nm is indicative of the octa-brominated porphyrin and the visible bands appeared at 684, and 631 nm in DCM.

### Example 9

### Synthesis of CuOHTCPBr (copper meso-5, 10, 15, 20-tetrakis [3-methoxy-4-o-hydroxylcarboranyl phenyl]octabromoporphyrin) (IX)

For this example, 205 mg (0.097 mmol) of CuOMTCPBr (the octabromoporphyrin compound (VIII) which was synthesized in Example 10) was dissolved in 40 mL of dichloromethane and stirred under nitrogen. 2.5 mL (2.5 mmol, 26 mol eq.) of boron tribromide (BBr) was added and the solution stirred for 30 minutes. The solution was cooled in an ice bath while an aqueous saturated sodium bicarbonate (40 mL) was added turning the red solution to green. The solution was then stirred for another 30 minutes.

The optical spectrum showed absorbances at 474, 586, and 638 nm in dichloromethane. When substituents on the phenyl rings of TPP are substituted, there is not a significant change in the optical spectrum because they do not involve bonds directly on the porphyrin ring. In contrast, the metal insertion or beta bromination reactions in the previous steps involve bond formation of atoms in the porphyrin skeleton and, therefore, directly affect the electronic energy states of the porphyrin pi system. This does show, however, that no unexpected change has occurred in the porphyrin ring structure. The solution was worked up with dichloromethane to extract CuOHTCPBr, which was then washed 3 times with water and dried over anhydrous sodium sulfate. The solvents were removed by rotary evaporation. The crude residue was purified using a silica pad (150 mL sintered glass funnel, 4 cm deep) and eluting with dichloromethane. 134 mg (0.065 mmol) of CuOHTCPBr was recovered giving a yield of 67%. The CuOHTCPBr product was 96% pure by HPLC.

### Animal Testing

The following examples include tests which were conducted to biologically evaluate CuOMTCPBr and CuOHTCPBr in mice, including biodistribution and toxicity testing. Subcutaneous (s.c.) EMT-6 tumors were initiated on the dorsal thorax of 18-22 g BALB/c mice (Taconic Farms, Germantown, N.Y.) using single-cell suspensions of 2.5×10⁵ cells in 0.05-0.10 mL culture medium. EMT-6 tumor cells were grown in vivo and in vitro in succession. Single-cell monolayers were prepared from mouse-grown tumors by trypsinization, expanded in alpha MEM (“minimum essential medium”) with 10% FBS (Gibco BRL Products, Grand Island, N.Y.) for several passages. Aliquots of the cells in 10% DMSO (dimethyl sulfoxide) were frozen in liquid nitrogen for storage and were thawed and regrown in tissue culture medium prior to implantation. Porphyrin injections were initiated 10 or 11 days after tumor cell implantation.

Porphyrin Formulation And Administration

CuOMTCPBr was formulated at a concentration of 5.0 mg/mL in 9% Cremophor EL and 18% propylene glycol in saline. Tumor-bearing mice were given CuOMTCPBr in either 2 or 4 intraperitoneal (i.p.) injections (i.e., injections which deliver the drug into the peritoneum) at 2 per day (with 8-hour interval) for a 200 or 400 mg/kg dose, respectively. Mice were euthanized 1, 2 or 3 days after the last injection.

CuOHTCPBr was formulated at a concentration of 3.5 mg/mL in 2% Cremophor ELP, 1% Tween 80, 2.2% polyethylene glycol 400 (PEG 400), and 4% absolute ethanol. Solid porphyrin and ethanol are added to a mixture of Cremophor and Tween 80 in a 70° C. bath. After about 1 hour of stirring, the PEG 400 is added and the mixture is allowed to stir overnight. After cooling to room temperature, water is added dropwise and the formulations are blended and filtered through a 0.2 μm filter. CuOHTCPBr was given in 3 i.p. injections over an 8-hour period for a total dose 143 mg/kg.

Euthanasia and Necropsy

Under deep Halothane inhalation anesthesia leading to euthanasia, right ventricular blood (0.2-0.5 ml total) was put into a Microtainer™ (Becton-Dickinson, Rutherford, N.J.) tube containing EDTA for hematological analyses, which was subsequently used for boron analyses. Tumor, brain, skin, liver, spleen, lungs, kidneys, heart, feces, small and large intestine were sampled at necropsy for boron analyses.

Boron Analyses

Direct current plasma-atomic emission spectroscopy (“DCP-AES”) was carried out using an ARL/Fisons Model SS-7 to assay boron with a detection limit of 0.1 μg B/ml. Samples from individual mice (50-130 mg) were digested at 60° C. with equal amounts of sulfuric acid and nitric acid (i.e., a 1:1 ratio). Triton X-100 and water were added to give final concentrations of about 50 mg tissue/mL, 15% total acid v/v and 5% Triton X-100 v/v. Tissue samples were analyzed from individual mice with microanalytical techniques for boron analysis using the 10B(n, α) ⁷Li reaction. Boron concentrations of injection solutions were determined by prompt gamma-ray spectroscopy, which was carried out at the Massachusetts Institute of Technology Reactor Prompt-Gamma Neutron Activation Facility.

Blood Analyses

Hematologic assays were carried out at Brookhaven National Laboratory using a VetScan HMT Hematology Analyzer, (Abaxis, Sunnyvale, Calif.). Mice were weighed daily and necropsies were carried out promptly after euthanasia. The percent weight changes use the body weights of the first day of administration as the initial weight.

### Example 10

Table 1 lists the boron concentrations (μg/g wet tissue) of various tissues from BALB/c mice bearing EMT-6 mammary carcinomas. The boron concentrations were measured at 2 or 3 days after about 400 mg/kg CuOMTCPBr (about 82 mg/kg B) were given in 4 i.p, injections over 2 days.

TABLE 1

Time after last injection

| | |
|---|---|
| 2 day | 3 days |

Number of mice

| | 6 | 6 |
|---|---|---|
| Tumor | 187 ± 44 | 142 ± 21 |
| Blood | 1.1 ± 0.6 | 0 ± 0.2 |
| Skin (pinna) | 17.1 ± 4.9 | 17.3 ± 2.0 |
| Brain | 0.2 ± 0.1 | 0 ± 0 |
| Liver | 716 ± 27 | 702 ± 47 |
| Spleen | 548 ± 145 | 498 ± 48 |
| Kidneys | 33.4 ± 7.7 | 33.9 ± 7.3 |
| Lungs | 30.6 ± 11.5 | 26.2 ± 3.8 |
| Heart | 47.3 ± 3.2 | 36.2 ± 2.4 |
| Small intestine | 78.1 ± 18.8 | 76.6 ± 18.7 |
| Large intestine | 44.5 ± 4.2 | 51.2 ± 15.5 |
| Feces | 9.3 ± 3.1 | 7.3 ± 3.1 |

CuOMTCPBr contains 20.4% boron as compared to 21.7% boron in CuTCPBr or 29.1% boron in CuOMTCPH. When CuOMTCPBr was given at a dose of 400 mg/kg, there was very high uptake of boron into the EMT-6 carcinoma (Table 1).

### Example 11

In this example, the boron uptake of three different porphyrin compounds was compared. Boron concentrations (mg/kg) in various tissues were arithmetically normalized to a boron dose of 64 mg/kg for mice given CuOMTCPBr or CuTCPBr and CuTCPH. The tissues were analyzed two days after the last injection. Table 2 shows that when the boron concentrations in tissues are arithmetically normalized to a constant boron dose, e.g. 64 mg/kg boron, the boron concentrations in tumor tissue of mice injected with CuOMTCPBr are significantly higher in comparison to mice injected with CuTCPH or CuTCPBr.

TABLE 2

| | Compound | | |
|---|---|---|---|
| | CuTCPH | CuTCPBr | CuOMTCPBr |
| Porphyrin dose | 200 | 293 | 312 |
| (mg/kg) | | | |
| Boron dose | 64 | 64 | 64 |
| (mg/kg) | | | |
| Number of mice | 5 | 5 | 6 |
| Tumor | 77.2 ± 19.6 | 107 | 146 |
| Blood | 0.5 ± 0.5 | 0.5 | 0.9 |
| Brain | 0 ± 0.1 | 0 | 0.2 |
| Skin (pinna) | 7.1 ± 3.3 | 9.7 | 13.3 |
| Liver | 427 ± 142 | 487 | 559 |
| Spleen | 193 ± 52 | 260 | 427 |
| Kidney | 9.6 ± 2.9 | 16.7 | 26.1 |
| Heart | 14.1 ± 2.1 | 21.3 | 36.9 |
| Lung | 14.1 ± 5.8 | 18.4 | 23.9 |

### Example 12

In this example, porphyrin concentrations (μg/g) in tissues were normalized to a porphyrin dose of 400 mg/kg for mice given CuOMTCPBr or CuTCPBr. The porphyrin concentrations were measured two days after the last injection. Table 3 shows porphyrin concentrations (for relevance in X-ray radiation therapy (XRT)) that are normalized to a porphyrin dose of 400 mg/kg.

TABLE 3

Compound

| | CuOMTCPBr | CuTCPBr |
|---|---|---|
| Number of mice | 6 | 5 |
| Tumor | 917 | 672 |
| Blood | 5.3 | 3.3 |
| Skin (pinna) | 84 | 61 |
| Brain | 1.0 | 0 |
| Liver | 3510 | 3060 |
| Spleen | 2690 | 1630 |
| Kidneys | 164 | 105 |
| Lungs | 150 | 116 |
| Heart | 232 | 134 |
| Small intestine | 383 | 380 |
| Large intestine | 218 | 241 |
| Feces | 46 | 57 |

As in Example 11, the tumor porphyrin concentrations from CuOMTCPBr are similarly higher than the tumor porphyrin concentrations from CuTCPBr.

### Example 13

This example compared hematological assays of blood from mice given 400 mg/kg CuOMTCPBr (n=6) and control mice given excipient only (n=3). Table 4 shows the results for hematological assays of blood from mice given 400 mg/kg CuOMTCPBr (n=6).

TABLE 4

| | Time after last injection | | |
|---|---|---|---|
| | 1 day | 2 days | 3 days |
| % Weight changes | −0.4 ± 1.9 | −1.2 ± 2.8 | −1.5 ± 1.4 |
| White blood | 13.6 ± 3.1 | 13.0 ± 1.7 | 11.9 ± 2.0 |
| count (m/m³) | | | |
| Platelet count | 1047 ± 255 | 985 ± 283 | 946 ± 91 |
| (m/m³) | | | |

Table 5 shows a hematological assay of blood from control mice given excipient only (n=3).

TABLE 5

| | Time after last injection | | |
|---|---|---|---|
| | 1 day | 2 days | 3 days |
| % Weight changes | 0 ± 2.7 | 2.1 ± 1.4 | 2.1 ± 2.8 |
| White blood | 6.4 ± 0.6 | 7.2 ± 1.2 | 5.3 ± 8.9 |
| count (m/m³) | | | |
| Platelet count | 903 ± 121 | 995 ± 74 | 833 ± 31 |
| (m/m³) | | | |

The weight change and hematology data shown in Tables 4 and 5 indicate that there is very little toxicity associated with CuOMTCPBr even at the high dose of 400 mg/kg. There is slightly greater weight loss in the porphyrin-injected mice versus those given excipient only. However, the platelet count, which is depressed in many porphyrins at half this dose, appears to be no different from the control mice that were given excipient only.

### Example 14

In this example, boron concentrations (μg/g wet tissue) of various tissues from BALB/c mice bearing EMT-6 mammary carcinomas were measured. Injections of 143 mg/kg CuOHTCPBr (about 30 mg/kg boron) were given in 4 i.p. injections over 2 days. The boron concentrations were measured at 1 or 2 days and are listed in Table 6.

TABLE 6

Time after last injection

| | |
|---|---|
| 1 day | 2 days |

Number of mice

| | 5 | 5 |
|---|---|---|
| Tumor | 56.8 ± 22.5 | 57.3 ± 13.6 |
| Blood | 0.8 ± 0.1 | 0.6 ± 0.1 |
| Skin (pinna) | 2.0 ± 1.0 | 3.5 ± 1.6 |
| Brain | 0.2 ± 0 | 0.1 ± 0 |
| Liver | 364 ± 65 | 362 ± 25 |
| Spleen | 362 ± 132 | 443 ± 76 |
| Kidneys | 11.4 ± 4.5 | 14.1 ± 3.3 |
| Lungs | 7.5 ± 3.7 | 10.5 ± 3.8 |
| Heart | 9.6 ± 2.7 | 13.4 ± 1.1 |
| Small intestine | 35.3 ± 10.1 | 25.0 ± 3.0 |
| Large intestine | 11.0 ± 2.0 | 9.9 ± 3.0 |
| Feces | 11.4 ± 3.6 | 9.7 ± 3.2 |

### Example 15

In this example, weight changes and hematological parameters were measured for mice given CuOHTCPBr (n=5) and excipient-only controls (n=3). The results are listed in Table 7.

TABLE 7

| | Compound | | | |
|---|---|---|---|---|
| | CuOHTCPBr | Control | CuOHTCPBr | Control |
| | Time after last injection (days) | | | |
| | 1 | 1 | 2 | 2 |
| % | −2.3 ± 1.2 | −0.3 ± 1.2 | 1.2 ± 1.6 | 0.6 ± 0.9 |
| Weight | | | | |
| change | | | | |
| Platelet | 1270 ± 125 | 1123 ± 95 | 1334 ± 304 | 1182 ± 143 |
| count | | | | |
| (m/m³) | | | | |
| White | 7.6 ± 1.2 | 4.7 ± 2.0 | 7.3 ± 0.6 | 4.8 ± 1.0 |
| blood | | | | |
| count | | | | |
| (m/m³) | | | | |

### Example 16

This example compared the percent of the injected dose of porphyrin or boron that remained in the tumor tissue 2 days after the last injection for CuOMTCPBr, CuOHTCPBr, CuTCPBr and CuTCPH. Table 8 shows the results in terms of the percentage of porphyrin or boron remaining in the tumor tissue per gram wet tissue.

TABLE 8

| Compound | % injected dose/g tumor tissue |
|---|---|
| CuOMTCPBr | 11.4 |
| CuOHTCPBr | 9.55 |
| CuTCPBr | 8.36 |
| CuTCPH | 6.07 |

The percentage of the injected boron/porphyrin the tumor per gram of tissue measured for CuOHTCPBr is slightly lower than that measured for CuOMTCPBr. However, the percentage of the injected boron/porphyrin for CuOHTCPBr is higher than that measured for CuTCPH or CuTCPBr. This indicates that, from a biodistribution standpoint only, CuOMTCPBr and CuOHTCPBr appear to be more efficient at targeting tumors for both BNCT and XRT. The toxicity from the latter is slightly less than that of the former but is likely attributable to the lower dose; CuOHTCPBr was given at 35% of the CuOMTCPBr dose.

From the data in Table 8, it can be concluded that CuOMTCPBr and CuOHTCPBr both have excellent biodistribution properties for both BNCT and XRT with minimal or no toxicity associated at very high porphyrin doses.

Thus, while there have been described the preferred embodiments of the present invention, those skilled in the art will realize that other embodiments can be made without departing from the spirit of the invention, which includes all such further modifications and changes as come within the true scope of the claims set forth herein.

1. A compound of the formula 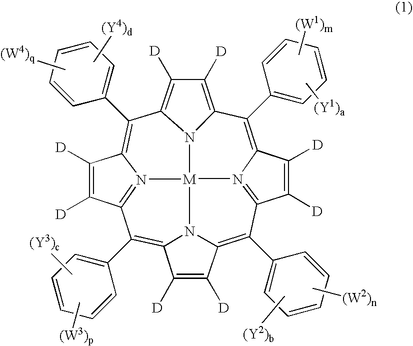
D is a halogen, a halogen isotope, a combination thereof or a combination thereof that includes from one to three hydrogen;
Y¹, Y², Y³ and Y⁴ are independently on the ortho, meta or para position on the phenyl rings, and are independently hydrogen, alkyl, cycloalkyl, aryl, alkylaryl, arylalkyl, heteroaryl, or an alkyl, cycloalkyl, aryl, alkylaryl, arylalkyl, or heteroaryl group substituted with 1 to 4 hydrophilic groups selected from hydroxy, alkoxy, —C(O)OR⁵, —SOR⁶, —SO₂R⁶, nitro, amido, ureido, carbamato, —SR⁷, —NR⁸R⁹ or poly-alkyleneoxide; or a substituent represented by the following formula:
—X—(CR¹R²)ᵣ-Z (2)
provided that at least two of (Y¹)ₐ, (Y²)_{b}, (Y³)_{c} and (Y⁴)_{d} are represented by formula (2);
X is oxygen or sulfur;
Z is a carborane cluster comprising at least two carbon atoms and at least three boron atoms, or at least one carbon atom and at least five boron atoms, within a cage structure;
r is 0 or an integer from 1 to 20;
W¹, W², W³ and W⁴ are independently hydrogen or hydrophilic groups selected from hydroxy, alkoxy, —C(O)OR⁵, —SOR⁶, —SO₂R⁶, nitro, amido, ureido, carbamato, —SR⁷, —NR⁸R⁹ or polyalkylene oxide;
R¹, R², R⁵, R⁶, R⁷, R⁸ and R⁹ are independently selected from hydrogen and C₁ to C₄ alkyl;
a, b, c and d independently represent an integer from 1 to 4;
m, n, p and q independently represent an integer from 1 to 4, provided that at least one of
m, n, p and q is not hydrogen, and each of the sums a+m, b+n, c+p and d+q, independently represents an integer from 1 to 5; and
M is either two hydrogen ions; a single monovalent metal ion; two monovalent metal ions; a divalent metal ion; a trivalent metal ion; a tetravalent metal ion; a pentavalent metal ion; a hexavalent metal ion; a radioactive metal ion useful in radioisotope-mediated radiation therapy or imageable by single photon emission computed tomography (SPECT) or positron emission tomography (PET); a paramagnetic metal ion detectable by magnetic resonance imaging (MRI); a metal ion suitable for boron neutron capture therapy (BNCT), X-ray radiation therapy (XRT) or photodynamic therapy (PDT); or a combination thereof; wherein when M is a single monovalent metal ion, the compound is charge-balanced by a counter cation; and when M is a trivalent, tetravalent, pentavalent, or hexavalent metal ion, the compound is charge-balanced by an appropriate number of counter anions, dianions, or trianions.

2. The compound according to claim 1, wherein Z is selected from the carboranes —C₂HB₉H₁₀ or —C₂HB₁₀H₁₀ wherein —C₂HB₉H₁₀ is nido ortho-, meta- or para-carborane, and —C₂HB₁₀H₁₀ is closo ortho-, meta- or para-carborane.

3. The compound according to claim 1, wherein M is vanadium, manganese, iron, ruthenium, technetium, chromium, platinum, cobalt, nickel, copper, zinc, germanium, indium, tin, yttrium, gold, barium, tungsten or gadolinium.

4. The compound according to claim 1, wherein a, b, c, and d are 1, and Y¹, Y², Y³, and Y⁴ are represented by —X—(CR¹R²)ᵣ-Z (formula 2).

5. The compound according to claim 4, wherein Z is selected from the carboranes —C₂HB₉H₁₀ or —C₂HB₁₀H₁₀, wherein —C₂HB₉H₁₀ is nido ortho-, meta- or para-carborane, and —C₂HB₁₀H₁₀ is closo ortho-, meta- or para-carborane.

6. The compound according to claim 5, wherein M is vanadium, manganese, iron, ruthenium, technetium, chromium, platinum, cobalt, nickel, copper, zinc, germanium, indium, tin, yttrium, gold, barium, tungsten or gadolinium.

7. The compound according to claim 6, wherein X is O; R¹ and R² are H; r is 1; and m, n, p and q are each 1.

8. The compound according to claim 7, wherein Y¹, Y², Y³ and Y⁴ are in the para position on the phenyl ring, and W¹, W², W³ and W⁴ are independently, hydroxy or alkoxy groups.

9. The compound according to claim 8, wherein the alkoxy groups are methoxy groups.

10. The compound according to claim 9, wherein the methoxy groups are in the meta position of the phenyl ring.

11. The compound according to claim 8, wherein the hydroxy groups are in the meta position of the phenyl ring.

12. The compound according to claim 1, wherein all of the D are halogens or halogen isotopes.

13. The compound according to claim 8, wherein all of the D are halogens or halogen isotopes.

14. The compound according to claim 8, wherein the halogen is bromine or iodine and the halogen isotope is a bromine isotope or an iodine isotope.

15. A method of bimodal cancer treatment in a subject comprising:
administering to the subject a composition comprising a compound according to claim 1; and
irradiating the subject.

16. The method according to claim 15, wherein said irradiation is by a method utilizing thermal or epithermal neutrons, X-rays or laser red light.

17. The method according to claim 15, wherein said bimodal cancer treatment comprises boron neutron capture therapy (BNCT), X-ray radiation therapy (XRT), photodynamic therapy (PDT), single photon emission computed tomography (SPECT), positron emission tomography (PET), wherein M is a SPECT- and/or PET-imageable radioactive metal ion, or magnetic resonance imaging (MRI), wherein M is a paramagnetic metal ion.

18. A method of bimodal cancer treatment in a subject comprising:
administering to the subject a composition comprising a compound according to claim 8; and
irradiating the subject.

19. The method according to claim 18, wherein said irradiation is by a method utilizing thermal or epithermal neutrons, X-rays or laser red light.

20. The method according to claim 19, wherein said bimodal cancer treatment comprises boron neutron capture therapy (BNCT), X-ray radiation therapy (XRT), photodynamic therapy (PDT), single photon emission computed tomography (SPECT), positron emission tomography (PET), wherein M is a SPECT- and/or PET-imageable radioactive metal ion, or magnetic resonance imaging (MRI), wherein M is a paramagnetic metal ion.

21. A method of imaging a tumor and surrounding tissue in a subject comprising:
administering to the subject a composition comprising a compound according to claim 3; and
observing the metal ion in the subject, thereby imaging the tumor and surrounding tissue, wherein said imaging is by a method selected from magnetic resonance imaging (MRI), single photon emission computed tomography (SPECT), or positron emission tomography (PET) methods.

22. A method of imaging a tumor and surrounding tissue in a subject comprising:
administering to the subject a composition comprising a compound according to claim 8; and
observing the metal ion in the subject, thereby imaging the tumor and surrounding tissue,
wherein said imaging is by a method selected from magnetic resonance imaging (MRI), single photon emission computed tomography (SPECT), or positron emission tomography (PET) methods.

23. A compound of the formula 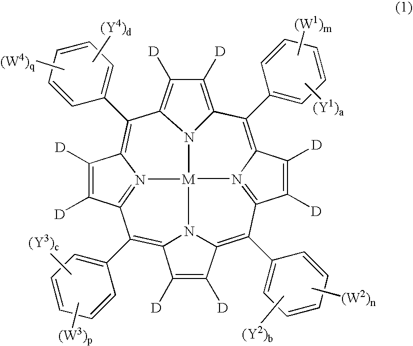
D is fluorine, a fluorine isotope, chlorine, a chlorine isotope, bromine, a bromine isotope, iodine, an iodine isotope, a combination thereof or a combination thereof that includes from one to three hydrogen;
Y¹, Y², Y³ and Y⁴ are independently on the ortho, meta or para position on the phenyl rings, and are independently hydrogen, alkyl, cycloalkyl, aryl, alkylaryl, arylalkyl, heteroaryl, or an alkyl, cycloalkyl, aryl, alkylaryl, arylalkyl, or heteroaryl group substituted with 1 to 4 hydrophilic groups selected from hydroxy, alkoxy, —C(O)OR⁵, —SOR⁶, —SO₂R⁶, nitro, amido, ureido, carbamato, —SR⁷, —NR⁸R⁹, or poly-alkyleneoxide, or a substituent represented by the following formula:
—X—(CR¹R²)ᵣ-Z (2)
provided that at least two of (Y¹)ₐ, (Y²)_{b} (Y³)_{c} and (Y⁴)_{d} are represented by formula (2);
X is oxygen or sulfur;
Z is a carborane cluster comprising at least two carbon atoms and at least three boron atoms, or at least one carbon atom and at least five boron atoms, within a cage structure;
r is 0 or an integer from 1 to 20;
W¹, W², W³, and W⁴ are independently hydrogen or hydrophilic groups selected from hydroxy, alkoxy, —C(O)OR⁵, —SOR⁶, —SO₂R⁶, nitro, amido, ureido, carbamato, —SR⁷, —NR⁸R⁹ or polyalkylene oxide;
R¹, R², R⁵, R⁶, R⁷, R⁸ and R⁹ are independently selected from hydrogen and C₁ to C₄ alkyl;
a, b, c and d independently represent an integer from 1 to 4;
m, n, p and q independently represent an integer from 1 to 4, provided that at least one of m, n, p and q is not hydrogen, and each of the sums a+m, b+n, c+p and d+q, independently represents an integer from 1 to 5;
M is a trivalent, tetravalent, pentavalent or hexavalent metal ion; and
wherein the porphyrin-metal complex is charge-balanced by one or more porphyrin compounds containing a divalent negative charge.

24. The compound according to claim 23, wherein said one or more porphyrin compounds containing a divalent negative charge are represented by the formula 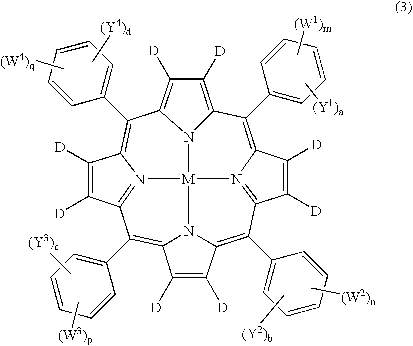
D is fluorine, a fluorine isotope, chlorine, a chlorine isotope, bromine, a bromine isotope, iodine, an iodine isotope, a combination thereof or a combination thereof that includes from one to three hydrogen;
Yhu 1, Y², Y³ and Y⁴ are independently on the ortho, meta or para position on the phenyl rings, and are independently hydrogen, alkyl, cycloalkyl, aryl, alkylaryl, arylalkyl, heteroaryl, or an alkyl, cycloalkyl, aryl, alkylaryl, arylalkyl, or heteroaryl group substituted with 1 to 4 hydrophilic groups selected from hydroxy, alkoxy, —C(O)OR⁵, —SOR⁶, —SO₂R⁶, nitro, amido, ureido, carbamato, —SR⁷, —NR⁸R⁹ or poly-alkyleneoxide, or a substituent represented by the following formula:
—X—(CR¹R²)ᵣ-Z (2)
provided that at least two of (Y¹)ₐ, (Y²)_{b}, (Y³)_{c} and (Y⁴)_{d} are represented by formula (2);
X is oxygen or sulfur;
Z is a carborane cluster comprising at least two carbon atoms and at least three boron atoms, or at least one carbon atom and at least five boron atoms, within a cage structure;
r is 0 or an integer from 1 to 20;
W¹, W², W³ and W⁴ are independently hydrogen or hydrophilic groups selected from hydroxy, alkoxy, —C(O)OR⁵, —SOR⁶, —SO₂R⁶, nitro, amido, ureido, carbamato, —SR⁷, —NR⁸R⁹ or polyalkylene oxide;
R¹, R², R⁵, R⁶, R⁷, R⁸ and R⁹ are independently selected from hydrogen and C₁ to C₄ alkyl;
a, b, c and d independently represent an integer from 1 to 4;
m, n, p and q independently represent an integer from 1 to 4; and
provided that at least one of m, n, p and q is not hydrogen, and each of the sums a+m, b+n, c+p and d+q, independently represents an integer from 1 to 5.

25. A method of imaging a tumor and surrounding tissue in a subject comprising:
administering a composition comprising a compound according to claim 23 to the subject; and
observing the metal ion in the subject, thereby imaging the tumor and surrounding tissue.

26. A method of bimodal cancer treatment in a subject comprising:
administering to the subject a composition comprising a compound according to claim 23; and
irradiating the subject.

27. The method according to claim 26, wherein said irradiation is by a method utilizing thermal or epithermal neutrons, X-rays or laser red light.

28. The method according to claim 26, wherein said bimodal cancer treatment comprises boron neutron capture therapy (BNCT), X-ray radiation therapy (XRT), photodynamic therapy (PDT), single photon emission computed tomography (SPECT), positron emission tomography (PET), wherein M is a SPECT- and/or PET-imageable radioactive metal ion, or magnetic resonance imaging (MRI), wherein M is a paramagnetic metal ion.

29. A compound of the formula 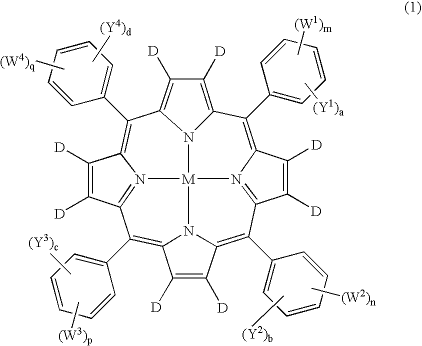
D is a halogen, a halogen isotope, a combination thereof or a combination thereof that includes from one to three hydrogen;
Y¹, Y², Y³ and Y⁴ are independently on the ortho, meta or para position on the phenyl rings, and are independently hydrogen, alkyl, cycloalkyl, aryl, alkylaryl, arylalkyl, heteroaryl, or an alkyl, cycloalkyl, aryl, alkylaryl, arylalkyl, or heteroaryl group substituted with 1 to 4 hydrophilic groups selected from hydroxy, alkoxy, —C(O)OR⁵, —SOR⁶, —SO₂R⁶, nitro, amido, ureido, carbamato, —SR⁷, —NR⁸R⁹ or poly-alkyleneoxide; or a substituent represented by the following formula:
—O—CH₂-Z (4)
provided that at least two of (Y¹)ₐ, (Y²)_{b}, (Y³)_{c} and (Y⁴)_{d} are represented by formula (4);
Z is a carborane cluster comprising at least two carbon atoms and at least three boron atoms, or at least one carbon atom and at least five boron atoms, within a cage structure;
W¹, W², W³ and W⁴ are independently hydrogen, a hydroxyl group or an alkoxy group;
R⁵, R⁶, R⁷, R⁸ and R⁹ are independently selected from hydrogen and C₁ to C₄ alkyl;
a, b, c and d independently represent an integer from 1 to 2;
m, n, p and q independently represent an integer from 1 to 2, provided that at least one of m, n, p and q is not hydrogen, and each of the sums a+m, b+n, c+p and d+q, independently represents an integer from 1 to 3; and
M is vanadium, manganese, iron, ruthenium, technetium, chromium, platinum, cobalt, nickel, copper, zinc, germanium, indium, tin, yttrium, gold, barium, tungsten or gadolinium.

30. The compound according to claim 29, wherein Z is selected from the carboranes —C₂HB₉H₁₀ or —C₂HB₁₀H₁₀, wherein —C₂HB₉H₁₀ is nido ortho-, meta- or para-carborane, and —C₂HB₁₀H₁₀ is closo ortho-, meta- or para-carborane.

31. The compound according to claim 29, wherein a, b, c, and d are 1, m, n, p and q are each 1 and Y¹, Y², Y³ and Y⁴ are independently hydrogen or are represented by —O—CH₂-Z (formula 4).

32. The compound according to claim 31, wherein Z is selected from the carboranes —C₂HB₉H₁₀ or —C₂HB₁₀H₁₀, wherein —C₂HB₉H₁₀ is nido ortho-, meta- or para-carborane, and —C₂HB₁₀H₁₀ is closo ortho-, meta- or para-carborane.

33. The compound according to claim 32, wherein Y¹, Y², Y³ and Y⁴ are in the para position on the phenyl ring, and W¹, W², W³ and W⁴ are in the meta position of the phenyl ring.

34. The compound according to claim 33 wherein all of the D are halogens or halogen isotopes.

35. The compound according to claim 34, wherein the halogen is bromine or iodine and the halogen isotope is a bromine isotope or an iodine isotope.

36. A method of bimodal cancer treatment in a subject comprising:
administering to the subject a composition comprising a compound according to claim 29; and
irradiating the subject.

37. The method according to claim 36, wherein said irradiation is by a method utilizing thermal or epithermal neutrons, X-rays or laser red light.

38. The method according to claim 36, wherein said bimodal cancer treatment comprises boron neutron capture therapy (BNCT), X-ray radiation therapy (XRT), photodynamic therapy (PDT), single photon emission computed tomography (SPECT), positron emission tomography (PET), wherein M is a SPECT- and/or PET-imageable radioactive metal ion, or magnetic resonance imaging (MRI), wherein M is a paramagnetic metal ion.

39. A method of imaging a tumor and surrounding tissue in a subject comprising:
administering to the subject a composition comprising a compound according to claim 29; and
observing the metal ion in the subject, thereby imaging the tumor and surrounding tissue,
wherein said imaging is by a method selected from magnetic resonance imaging (MRI), single photon emission computed tomography (SPECT), or positron emission tomography (PET) methods.

40. A compound of the formula 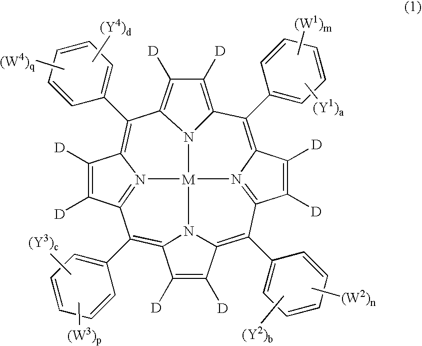
D is a halogen, a halogen isotope, a combination thereof or a combination thereof that includes from one to three hydrogen;
Y¹, Y², Y³ and Y⁴ are independently on the ortho, meta or para position on the phenyl rings, and are independently hydrogen, hydroxyl groups, alkoxy groups or a substituent represented by the following formula:
—O—CH₂-Z (4)
provided that at least two of (Y¹)ₐ, (Y²)_{b}, (Y³)_{c} and (Y⁴)_{d} are represented by formula (4);
Z is selected from the carboranes —C₂HB₉H₁₀ or —C₂HB₁₀H₁₀, wherein —C₂HB₉H₁₀ is nido ortho-, meta- or para-carborane, and —C₂HB₁₀H₁₀ is closo ortho-, meta- or para-carborane;
W¹, W², W³ and W⁴ are independently hydrogen, a hydroxyl group or an alkoxy group;
a, b, c and d independently represent an integer from 1 to 2;
m, n, p and q independently represent an integer from 1 to 2, provided that at least one of m, n, p and q is not hydrogen, and each of the sums a+m, b+n, c+p and d+q, independently represents an integer from 1 to 3; and
M is vanadium, manganese, iron, ruthenium, technetium, chromium, platinum, cobalt, nickel, copper, zinc, germanium, indium, tin, yttrium, gold, barium, tungsten or gadolinium.

41. The compound according to claim 40, wherein a, b, c, and d are 1, m, n, p and q are each 1 and Y¹, Y², Y³ and Y⁴ independently are hydrogen or are represented by —O—CH₂-Z (formula 4).

42. The compound according to claim 41, wherein M is manganese, nickel, copper, zinc or gadolinium, Y¹, Y², Y³ and Y⁴ are in the para position on the phenyl ring, W¹, W², W³ and W⁴ are in the meta position of the phenyl ring and all of the D are halogens or halogen isotopes.

43. The compound according to claim 42, wherein the halogen is bromine or iodine and the halogen isotope is a bromine isotope or an iodine isotope.

44. A method of bimodal cancer treatment in a subject comprising:
administering to the subject a composition comprising a compound according to claim 40; and
irradiating the subject.

45. The method according to claim 44, wherein said irradiation is by a method utilizing thermal or epithermal neutrons, X-rays or laser red light.

46. The method according to claim 44, wherein said bimodal cancer treatment comprises boron neutron capture therapy (BNCT), X-ray radiation therapy (XRT), photodynamic therapy (PDT), single photon emission computed tomography (SPECT), positron emission tomography (PET), wherein M is a SPECT- and/or PET-imageable radioactive metal ion, or magnetic resonance imaging (MRI), wherein M is a paramagnetic metal ion.

47. A method of imaging a tumor and surrounding tissue in a subject comprising:
administering to the subject a composition comprising a compound according to claim 40; and
observing the metal ion in the subject, thereby imaging the tumor and surrounding tissue, wherein said imaging is by a method selected from magnetic resonance imaging (MRI), single photon emission computed tomography (SPECT), or positron emission tomography (PET) methods.
